(19) United States
(12) Patent Application Publication
(10) Pub. No.: US 2007/0185116 A1
(43) Pub. Date: Aug. 9, 2007

(54) Bicyclic compounds

(75) Inventors: Miki Hirai, Osaka-Shi (JP); Mari Kusama, Osaka-Shi (JP); Toshihiro Hosaka, Osaka-Shi (JP); Shuntarou Kohnomi, Matsuyama-Shi (JP)

Correspondence Address: BIRCH STEWART KOLASCH & BIRCH, PO BOX 747, FALLS CHURCH, VA, 22040-0747, US

(73) Assignee: TANABE SEIYAKU CO., LTD., Osaka (JP)

(21) Appl. No.: 11/597,890

(22) PCT Filed: May 30, 2005
(86) PCT No.: PCT/JP05/10287

§ 371 Date: Nov. 29, 2006

Related U.S. Application Data
(60) Provisional application No. 60/584,142, filed on Jul. 1, 2004.

(30) Foreign Application Priority Data
May 31, 2004 (JP) 2004-160660
Jun. 29, 2004 (JP) 2004-191849
Dec. 1, 2004 (JP) 2004-348136

(51) Int. Cl. A61K 31/44; A61K 31/505; C07D 213/02; C07D 239/24

(52) U.S. Cl. 514/247; 514/277; 544/242; 544/335; 546/1; 546/336

(57) ABSTRACT

The present invention is to provide a bicyclic compound represented by the following formula: 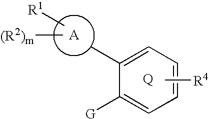 wherein Ring Q is pyridine or pyrimidine; Ring A is benzene or a heterocyclic ring;
G is Ring B optionally having a substituent(s) R³, or an amino optionally substituted by one or two selected from the group consisting of alkyl(s), aralkyl(s) and cycloalkyl(s); Ring B is benzene, a heterocyclic ring, a cycloalkane or a cycloalkene;
R¹ is a group selected from the following formulae: 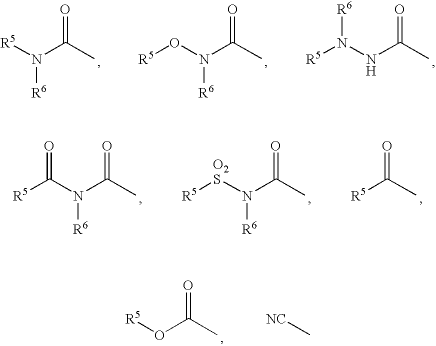
R² and R³ may be the same or different from each other, and each is cyano, nitro, etc.; m is 0, 1 or 2;
R⁴ is hydrogen, a halogen, etc.; and
R⁵ and R⁶ may be the same or different from each other, and each is hydrogen, an optionally substituted alkyl, etc., or a pharmaceutically acceptable salt thereof, which is a large conductance calcium-activated K channel opener useful for treatment of pollakiuria, urinary incontinence, etc.

### TECHNICAL FIELD

This invention relates to a large conductance calcium-activated K channel opener, which is useful for treatment of disorders or diseases such as pollakiuria, urinary incontinence, asthma, chronic obstructive pulmonary diseases (COPD), cerebral infarction, subarachnoid hemorrhage, and the like.

### BACKGROUND ART

Potassium is the most abundant intracelluar cation, and is very important in maintaining physiological homeostasis. Potassium channels are present in almost all vertebrate cells, and the potassium influx through these channels is indispensable for maintaining hyperpolarized resting membrane potential. Large conductance calcium activated potassium channels (also BK channels or maxi-K channels) are expressed especially in neurons and smooth muscle cells. Because both of the increase of intracellular calcium concentration and membrane depolarization can activate maxi-K channels, maxi-K channels have been thought to play a pivotal role in regulating voltage-dependent calcium influx. Increase in the intracellular calcium concentration mediates many processes such as release of neurotransmitters, contraction of smooth muscles, cell growth and death, and the like. Actually, the opening of maxi-K channels causes strong membrane hyperpolarization, and inhibits these calcium-induced responses thereby. Accordingly, by inhibiting various depolarization-mediated physiological responses, a substance having an activity of opening maxi-K channels is useful for the treatment of diseases such as cerebral infarction, subarachnoid hemorrhage, pollakiuria, urinary incontinence, and the like.

There has been a report that a medicine which opens a BK channel has an activity to inhibit electrically induced contraction of respiratory tract preparation of guinea pig (Non patent publication 1). Therefore, it is effective for treatment of, for example, asthma, COPD, etc. Also, there has been disclosed that a medicine which opens a BK channel can be an agent for treatment of sexual function disorder such as erectile dysfunction, etc. (Patent publication 1).

There have been various reports on a large conductance calcium-activated potassium channel opener. For example, a pyrrole derivative (Patent publication 2), a furan derivative (Patent publication 3), a nitrogen-containing 5-membered ring derivative in which the nitrogen is substituted by phenyl or benzyl (Patent publication 4), a diphenyltriazole derivative (Non patent publication 2), a celecoxib derivative (Patent publication 5), etc. have been reported.

General synthetic method of pyrimidine derivatives such as 4-amino-5-(4-cyanophenyl)pyrimidine is disclosed in Non patent publication 3.

2-(4-fluorophenyl)-3-(4-pyrimidyl)pyridine derivatives are disclosed in Patent publication 6 as active ingredients for treating a CSBP/RK/p38 kinase mediated disease.

[Patent publication 1] WO 00/34244

[Patent publication 2] WO 96/40634

[Patent publication 3] JP 2000-351773

[Patent publication 4] WO 98/04135

[Patent publication 5] EP 1400243

[Patent publication 6] WO 00/40243

[Non-Patent publication 1] J. Pharmacol. Exp. Ther., (1998) 286: 952-958

[Non-Patent publication 2] J. Med. Chem., Vol. 45, p. 2942-2952(2002)

[Non-Patent publication 3] Anales de la Asociacion Quimica Argentina, 56(1-2), 73(1968)

### SUMMARY OF THE INVENTION

An object of the present invention is to provide a compound having an excellent large conductance calcium-activated K channel opening activity while having less side effects, and useful for the treatment of diseases such as pollakiuria, urinary incontinence, asthma, COPD, cerebral infarction, subarachnoid hemorrhage, and the like.

The present inventors have studied intensively to achieve the above-mentioned objects, and as a result, they have found that the bicyclic compounds of the following formula have an excellent large conductance calcium-activated K channel opening activity, whereby they have accomplished the present invention.

That is, the present invention is described as follows:
[1] A bicyclic compound of formula (1): 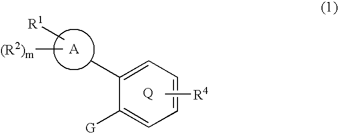
wherein Ring Q is pyridine or pyrimidine; Ring A is benzene or a heteroaromatic ring; 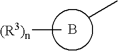
or an amino optionally substituted by one or two selected from the group consisting of alkyl(s), aralkyl(s) and cycloalkyl(s);
Ring B is benzene, a heterocyclic ring, a cycloalkane or a cycloalkene;
R¹ is a group selected from the following formulae: 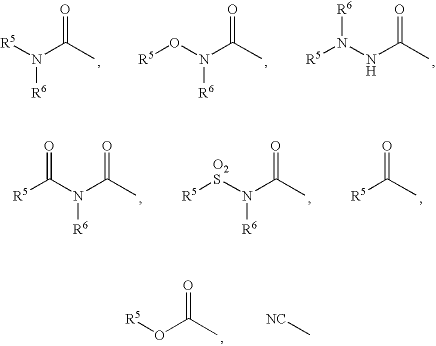
R² and R³ may be the same or different from each other, and each is cyano, nitro, hydroxyl, an alkoxy, a halogen, carboxyl, an alkoxycarbonyl, an optionally substituted carbamoyl, an optionally substituted amino or optionally substituted alkyl; provided that when m is 2, two R²s may be the same or different from each other, and when n is 2, two R³s may be the same or different from each other;
m and n may be the same or different from each other, and each is 0, 1 or 2;
R⁴ is hydrogen, a halogen, cyano, an alkoxy, hydroxyl, carbamoyl, an optionally substituted amino, an optionally substituted alkyl, an optionally substituted aryloxy, a cycloalkyloxy or an optionally substituted heterocyclic group; and
R⁵ and R⁶ may be the same or different from each other, and each is hydrogen, an optionally substituted alkyl, an optionally substituted cycloalkyl where the cycloalkyl may be fused with an aryl, an optionally substituted aryl, an optionally substituted heterocyclic group, or an alkoxycarbonyl, or R⁵ and R⁶ may form an optionally substituted heterocyclic ring in combination with atoms to which they are bonded,

excluding 4-amino-5-(4-cyanophenyl)pyrimidine, or a pharmaceutically acceptable salt thereof.
[2] The bicyclic compound or a pharmaceutically acceptable salt thereof of section [1], which compound is a compound of formula (1a): 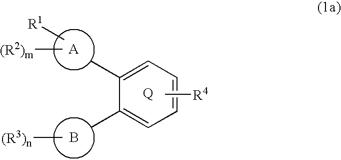
wherein Ring Q, Ring A, Ring B, R¹, R², R³, R⁴, m and
n have the same meanings as defined above.
   [3] The bicyclic compound or a pharmaceutically acceptable salt thereof of section [1] or [2], wherein the Ring Q is pyridine.
   [4] The bicyclic compound or a pharmaceutically acceptable salt thereof of section [2], which compound is a compound of formula (1b): 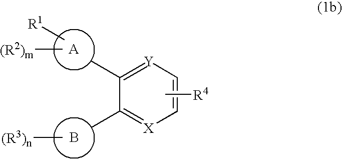
wherein one of X and Y is nitrogen, and the other is methine, and
Ring A, Ring B, R¹, R², R³, R⁴, m and n have the same meanings as defined above.
   [5] The bicyclic compound or a pharmaceutically acceptable salt thereof according to any one of sections [1] to [4], wherein Ring A is a 5- or 6-membered ring.
   [6] The bicyclic compound or a pharmaceutically acceptable salt thereof of any one of sections [1] to [4], wherein the Ring A is benzene, pyridine, pyrimidine or thiophene.
   [7] The bicyclic compound or a pharmaceutically acceptable salt thereof according to any one of sections [1] to [4], wherein the Ring A is benzene.
   [8] The bicyclic compound or a pharmaceutically acceptable salt thereof according to any one of sections [1] to [7], wherein Ring A is a 6-membered ring and R¹ is bonded to Ring A at the para-position to Ring Q
   [9] The bicyclic compound or a pharmaceutically acceptable salt thereof of any one of sections [1] to [8], wherein the Ring B is benzene, pyridine, pyrimidine, thiophene, piperidine, morpholine, cyclohexane, cyclohexene, pyrrolidine or pyrrole.
   [10] The bicyclic compound or a pharmaceutically acceptable salt thereof of any one of sections [1] to [9], wherein R¹ is a group selected from the following formulae:

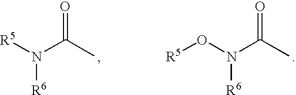

[11] The bicyclic compound or a pharmaceutically acceptable salt thereof of section [10], wherein R⁵ is hydrogen, an optionally substituted alkyl (wherein substituent(s) for the substituted alkyl are 1 to 3 groups selected from the following formulae), an optionally substituted cycloalkyl (said cycloalkyl may be fused with an aryl), an optionally substituted aryl or an optionally substituted heterocyclic group, and R⁶ is hydrogen, an alkoxycarbonyl, or an alkyl optionally substituted by hydroxyl(s) or alkoxy(s), or R⁵ and R⁶ may form an optionally substituted heterocyclic ring in combination with atom(s) to which they are bonded, 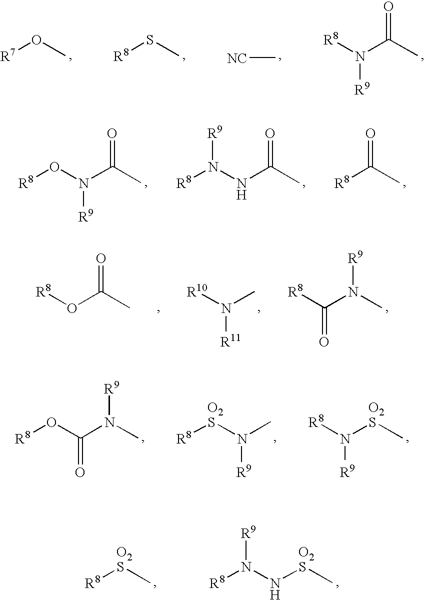
optionally substituted heterocyclic group, optionally substituted aryl,
wherein R⁷ is (1) hydrogen, (2) an alkyl optionally substituted by an optionally substituted aryl or an optionally substituted heterocyclic group, (3) a hydroxyalkyl, (4) an alkoxyalkyl or (5) an optionally substituted heterocyclic group;
R⁸ and R⁹ may be the same or different from each other, and each is (1) hydrogen, (2) an alkyl optionally substituted by an optionally substituted aryl or an optionally substituted heterocyclic group, (3) a hydroxyalkyl, (4) an alkoxyalkyl, (5) an alkoxycarbonyl, (6) an optionally substituted heterocyclic group or (7) an optionally substituted aryl or (8) R⁸ and R⁹ may form an optionally substituted heterocyclic ring in combination with atoms to which they are bonded; and
R¹⁰ and R¹¹ may be the same or different from each other, and each is (1) hydrogen, (2) an alkyl optionally substituted by an optionally substituted aryl or optionally substituted heterocyclic group, (3) a hydroxyalkyl, (4) an alkoxyalkyl, (5) an alkanoyl, (6) an alkylsulfonyl, (7) an alkoxycarbonyl or (8) an optionally substituted heterocyclic group.
   [12] The bicyclic compound or a pharmaceutically acceptable salt thereof of section [11], wherein the substituent(s) for the substituted alkyl of R⁵ are 1 to 3 groups selected from the following formulae: 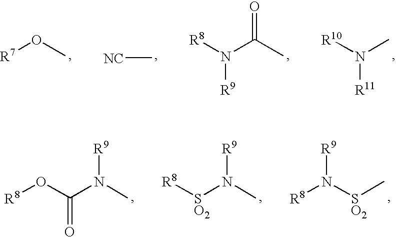
   optionally substituted heterocyclic group, optionally substituted aryl,
wherein R⁷, R⁸, R⁹, R¹⁰ and R¹¹ have the same meanings as defined above.

[13] The bicyclic compound or a pharmaceutically acceptable salt thereof of section [11], wherein the substituent(s) for the substituted alkyl of R⁵ are 1 to 3 groups selected from the following formulae: 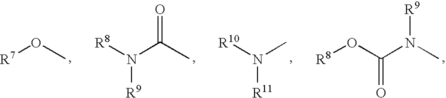
optionally substituted heterocyclic group, optionally substituted aryl,
wherein R⁷, R⁸, R⁹, R¹⁰ and R¹¹ have the same meanings as defined above.

The bicyclic compound or a pharmaceutically acceptable salt thereof according to any one of [1] to [13], wherein Ring Q is pyrimidine and R¹ is a group selected from the following formulae: 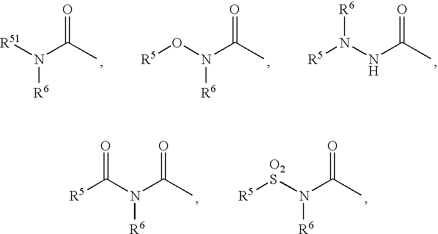
wherein R⁵ and R⁶ have the same meanings as defined in section [1], R⁵¹ is an alkyl substituted by 1 to 3 groups selected from the following formulae: 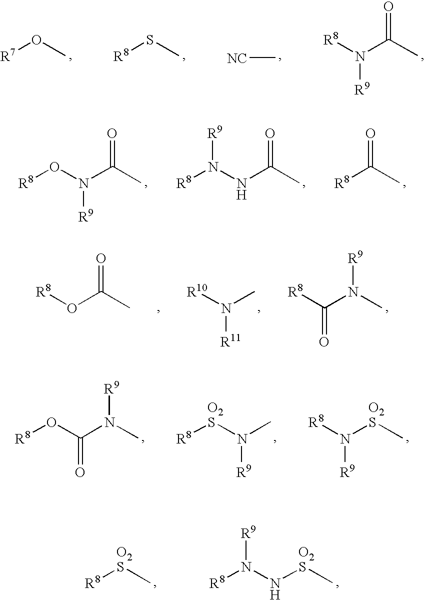
wherein R⁷, R⁸, R⁹, R¹⁰ and R¹¹ have the same meanings as defined in section [11].
   [15] The bicyclic compound or a pharmaceutically acceptable salt thereof of any one of [1] to [14], wherein m and n may be the same or different from each other, and each is 0 or 1.
   [16] The bicyclic compound or a pharmaceutically acceptable salt thereof of any one of [1] to [15], wherein R² and R³ may be the same or different from each other, and each is cyano, hydroxyl, an alkoxy, a halogen or an optionally substituted alkyl.
   [17] The bicyclic compound or a pharmaceutically acceptable salt thereof of any one of [1] to [16], wherein R⁴ is hydrogen, a halogen, or an optionally substituted alkyl.

[18] A medicine comprising the bicyclic compound or a pharmaceutically acceptable salt thereof of any one of sections [1] to [17].

[19] The medicine of section [18], which is a large conductance calcium-activated K channel opener.

[20] The medicine of [18], which is for the prophylaxis and/or treatment of pollakiuria, urinary incontinence, asthma or chronic obstructive pulmonary diseases.

[21] The medicine according to section [20], which is for the prophylaxis and/or treatment of pollakiuria, urinary incontinence or chronic obstructive pulmonary diseases.

[22] The bicyclic compound or a pharmaceutically acceptable salt thereof of any one of [1] to [17], wherein when Ring Q is 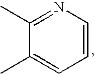
Ring B is neither

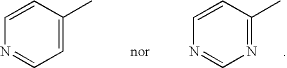

### DESCRIPTION OF THE PREFERRED EMBODIMENTS

Hereinafter, each group represented by the respective symbols in the present specification will be explained.

“Alkyl” and the alkyl in “alkoxyalkyl” and “alkylsulfonyl” are exemplified by a straight or branched C₁₋₆ alkyl, preferably by a straight or branched C₁₋₄ alkyl, and more specifically by methyl, ethyl, propyl, isopropyl, butyl, isobutyl, 1-methylpropyl, pentyl, hexyl, etc.

“Hydroxyalkyl” is exemplified by a straight or branched C₁₋₆ alkyl, preferably by a straight or branched C₁₋₄ alkyl, which is substituted by hydroxyl(s), and more specifically by hydroxymethyl, 2-hydroxyethyl, 3-hydroxypropyl, 2-hydroxypropyl, 3-hydroxybutyl, 4-hydroxybutyl, etc.

“Alkoxy” and the alkoxy in “alkoxyalkyl” and “alkoxycarbonyl” are exemplified by a straight or branched C₁₋₆ alkoxy, preferably by a straight or branched C₁₋₄ alkoxy, and more specifically by methoxy, ethoxy, propoxy, isopropoxy, butoxy, isobutoxy, tert-butoxy, pentyloxy, hexyloxy, etc.

“Halogen” includes fluorine, chlorine, bromine, and iodine.

“Alkanoyl” is exemplified by a straight or branched C₁₋₆ alkanoyl, preferably by a straight or branched C₁₋₄ alkanoyl, and more specifically by formyl, acetyl, propionyl, butyryl, pentanoyl, hexanoyl, etc.

“Haloalkyl” is exemplified by a straight or branched C₁₋₆ alkyl, preferably a straight or branched C₁₋₄ alkyl, which is substituted by halogen(s), and more specifically by chloromethyl, dichloromethyl, fluoromethyl, difluoromethyl, trifluoromethyl, 2,2,2-trifluoroethyl, 3-chloropropyl, 3-fluoropropyl, 4-chlorobutyl, 4-fluorobutyl, etc.

“Haloalkoxy” is exemplified by a straight or branched C₁₋₆ alkoxy, preferably a straight or branched C₁₋₄ alkoxy, which is substituted by halogen(s), and more specifically by chloromethoxy, dichloromethoxy, fluoromethoxy, difluoromethoxy, trifluoromethoxy, 2,2,2-trifluoroethoxy, perfluoroethoxy, 3-chloropropoxy, 3-fluoropropoxy, 4-chlorobutoxy, 4-fluorobutoxy, etc.

“Alkenyl” is exemplified by a straight or branched C₂₋₆ alkenyl, preferably by a straight or branched C₂₋₄ alkenyl, and more specifically by vinyl, allyl, 1-methyl-2-propenyl, 3-butenyl, 2-pentenyl, 3-hexenyl, etc.

“Aryl” and the aryl in “aryloxy” are exemplified by a moncyclic, bicyclic or tricyclic C₆₋₁₄ aryl, preferably by a C₆₋₁₀ aryl, and more specifically by phenyl, naphthyl, phenanthryl, anthryl, etc. Phenyl and naphthyl are particularly preferred.

“Aralkyl” is exemplified by a straight or branched C₁₋₆ alkyl, preferably by a straight or branched C₁₋₄ alkyl, which is substituted by aryl(s), more specifically by benzyl, 2-phenylethyl, 1-phenylethyl, 3-phenylpropyl, etc, further specifically by benzyl.

“Cycloalkyl” and the cycloalkyl in “cycloalkyloxy” are exemplified by a C₃₋₈ cycloalkyl, preferably by a C₃₋₆ cycloalkyl, and more specifically by cyclopropyl, cyclobutyl, cyclopentyl, cyclohexyl, etc. “Cycloalkyl fused with an aryl” is exemplified by a C₃₋₈ cycloalkyl, preferably by a C₃₋₆ cycloalkyl, which is fused with an aryl (preferably phenyl), and more specifically by indanyl, tetranyl, etc. The “cycloalkyl” and the “cycloalkyl fused with an aryl” may have substituent(s) which are exemplified by hydroxyl, a halogen, a C₁₋₄ alkyl, a C₁₋₄ alkoxy, etc., and preferably by hydroxyl. Specific examples for the substituted cycloalkyl fused with an optionally substituted aryl include 2-hydroxyindan-1-yl, etc.

“Cycloalkane” is exemplified by a C₃₋₈ cycloalkane, preferably by a C₃₋₆ cycloalkane, and more specifically by cyclopropane, cyclobutane, cyclopentane, cyclohexane, etc.

“Cycloalkene” is exemplified by a C₃₋₈ cycloalkene, preferably by a C₃₋₆ cycloalkene, and more specifically by cyclopropene, cyclobutene, cyclopentene, cyclohexene, etc.

“Heterocyclic group” is exemplified by a monocyclic or bicyclic 5 to 10-membered heterocyclic group, which may be partially or wholly saturated, containing 1 to 4 hetero atom(s) selected from nitrogen, oxygen and sulfur. The monocyclic or bicyclic heterocyclic group, which may be partially or wholly saturated, may be optionally substituted oxo.

The monocyclic heterocyclic group is preferably exemplified by a 5 to 7-membered heterocyclic group which may be partially or wholly saturated, containing 1 to 4 hetero atom(s) selected from nitrogen, oxygen and sulfur. It is specifically exemplified by oxazolyl, pyrrolidinyl, pyrrolyl, pyrazolyl, pyridyl, pyrimidinyl, pyrazinyl, tetrazolyl, thiazolyl, piperidyl, piperazinyl, morpholyl, tetrahydropyranyl, tetrahydrofuryl, imidazolidinyl, oxazolidinyl, etc.

The bicyclic heterocyclic group is preferably exemplified by a bicyclic heterocyclic group in which two of the same or different monocyclic heterocyclic groups above are fused, or a bicyclic heterocyclic group in which the above monocyclic heterocyclic group and benzene are fused. It is specifically exemplified by dihydroindolyl, tetrahydroquinolyl, etc.

On Ring Q, nitrogen(s) may be located at any position(s) as long as G and Ring A can be bonded to ring Q, and preferred are as followed:

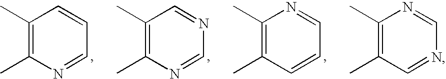

“Heteroaromatic ring” of Ring A is exemplified by a monocyclic or bicyclic 5- to 10-membered heteroaromatic ring containing 1 to 4 hetero atom(s) selected from nitrogen, oxygen and sulfur, and preferably exemplified by a 5- or 6-membered heteroaromatic ring. Specific examples thereof include thiophene, furan, pyrrole, pyridine, pyrimidine, pyrazine, benzo[b]thiophene, oxazole, isoxazole, thiazole, benzo[b]furan and quinoline. Preferred are pyridine, pyrimidine, and thiophene, and particularly preferred is pyridine.

“Heterocyclic ring” of Ring B is exemplified by a monocyclic or bicyclic 5- to 10-membered heterocyclic ring, which may be partially or wholly saturated, containing 1 to 4 hetero atom(s) selected from nitrogen, oxygen and sulfur, and preferably exemplified by a 5-membered heterocyclic ring which does not contains more than one nitrogen and a 6-membered heterocyclic ring. More preferably exemplified by a 6-membered aromatic heterocyclic ring. Specific examples thereof include thiophene, furan, pyrrole, pyridine, pyrimidine, pyrazine, piperidine, piperazine, pyrrolidine, tetrahydropyrane, benzo[b]thiophene, oxazole, isoxazole, thiazole, benzo[b]furan, 2,3-dihydroindole, 2,3-dihydrobenzo[b]furan, 1,4-benzodioxane, quinoline, pyrrolidine, morpholine, thiomorpholine, homopiperidine and 1,5-benzodioxepine. Preferred are pyridine, pyrimidine, and thiophene, and particularly preferred is pyridine in which Ring Q may be positioned at any position, and Preferred are at meta- or ortho-position from the position of nitrogen located on Ring B.

“Heterocyclic ring formed by R⁵ and R⁶ in combination with atom(s) to which they are bonded” and “heterocyclic ring formed by R⁸ and R⁹ in combination with atom(s) to which they are bonded” are exemplified by a saturated 5- to 8-membered monocyclic heterocyclic ring, containing one or two hetero atom(s) (such as nitrogen, oxygen, sulfur, etc.). Specific examples thereof include pyrrolidine, piperidine, piperazine, morpholine, thiomorpholine, homopiperidine, etc.

The “heterocyclic ring” may be substituted, and the substituents are exemplified by (1) an alkyl which may be optionally substituted by group(s) selected from (i) a halogen, (ii) hydroxyl, (iii) a haloalkoxy, (iv) an alkoxy which may be optionally substituted by halogen, alkyl(s), phenyl, etc., (v) carbamoyl which may be optionally substituted by alkyl(s), etc., (vi) cyano, (vii) an alkoxycarbonyl, (viii) carboxy, (ix) an amino which may be optionally substituted by alkyl(s), phenyl, etc., (x) an imino which may be optionally substituted by an alkoxy, hydroxyl, etc., and (xi) a heterocyclic group; (2) cyano; (3) a halogen; (4) an amino which may be optionally substituted by alkyl(s), an alkanoyl, a cycloalkyl, etc.; (5) an alkenyl; (6) an imino which may be optionally substituted by an alkoxy, hydroxyl, etc.; (7) a carbamoyl which may be optionally substituted by alkyl(s), aralkyl(s), etc.; (8) an alkoxycarbonyl; (9) a heterocyclic group; (10) oxo; etc. Preferred examples of the substituent(s) therefor include an alkyl optionally substituted by hydroxyl(s), and a 5- or 6-membered monocyclic heterocyclic group which may have 1 to 3 hetero atom(s) selected from nitrogen, oxygen and sulfur, and particularly preferably hydroxymethyl and pyrimidyl.

The “heterocyclic group” of R⁵ to R¹¹, and the “heterocyclic group” as a substituent for the substituted alkyl of R⁵ to R¹¹ are preferably exemplified by pyridyl, pyrazolyl, pyrazinyl, pyrimidinyl, tetrazolyl, tetrahydropyranyl, thiazolyl, piperidyl, morpholinyl, oxazolyl, piperazinyl, etc. The substituent for the heterocyclic group is exemplified by alkyl, haloalkyl, hydroxyl, alkoxy, etc., preferably by methyl, trifluoromethyl, hydroxyl, methoxy, etc. Particularly preferred examples of the heterocyclic group of R⁷ are pyrimidyl and tetrahydropyranyl. Particularly preferred example of the heterocyclic group of R¹⁰ and R¹¹ is pyridyl.

The substituent for the substituted alkyl of R⁵ and R⁶ is exemplified by a group selected from the following formulae, and the alkyl may be substituted by 1 to 3 groups(s) which may be the same or different: 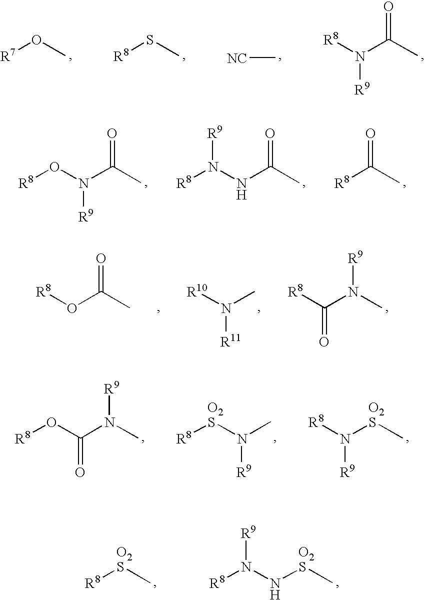
optionally substituted heterocyclic group, optionally substituted aryl,
wherein R⁷, R⁸, R⁹, R¹⁰ and R¹¹ have the same meanings as defined above.

Of these groups, preferred examples are: 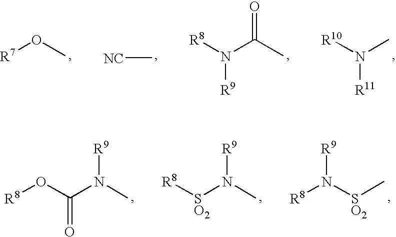
optionally substituted heterocyclic group, optionally substituted aryl,
etc., more preferably, 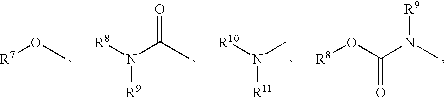
optionally substituted heterocyclic group, optionally substituted aryl, etc.

Specific examples of substituted alkyls for R⁵ or R⁶ include a group selected from the following formulae: 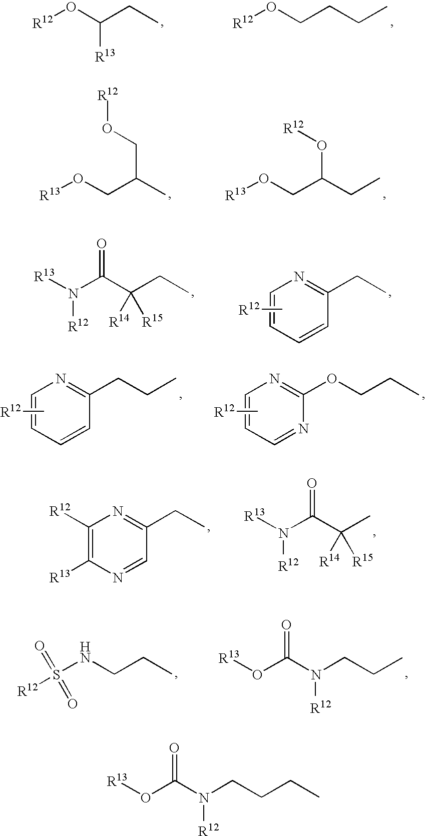
wherein R¹², R¹³, R¹⁴ and R¹⁵ may be the same or different from each other, and each is hydrogen or an alkyl, etc.

The substituent for the substituted aryl of R⁵ to R¹¹ is each exemplified by a halogen, hydroxyl, an alkoxy, an alkyl, a haloalkyl, etc.

The substituent for the substituted carbamoyl of R² and R³ is each exemplified by an alkyl-optionally substituted by a halogen, hydroxyl, an alkoxy, amino, a mono- or di-alkylamino, etc.

The alkyls in “an amino substituted by one or two selected from the group consisting of alkyl(s), aralkyl(s) and cycloalkyl(s)” of G are exemplified by a straight or branched C₁₋₆ alkyl, preferably by a branched C₁₋₄ alkyl, and more specifically by isopropyl, isobutyl, 1-methylpropyl, isoamyl, etc. Preferred is iso-propyl.

The substituent for the substituted amino of R², R³ and R⁴ is each exemplified by an alkyl optionally substituted by a halogen, hydroxyl, an alkoxy, amino, a mono- or di-alkylamino, etc.

The substituent for the substituted alkyl of R², R³ and R⁴ is each exemplified by hydroxyl, an alkoxy, a halogen, etc., and specific examples of the substituted alkyl may include hydroxymethyl, 2-hydroxyethyl, methoxymethyl, trifluoromethyl, etc.

Specific examples of R¹ to R¹³, G, Ring A and Ring B include a corresponding group in each compound described in the examples.

Examples of the pharmaceutically acceptable salts of the bicyclic compound (1) of the present invention may include, for example, inorganic acid salts such as hydrochloride, sulfate, phosphate or hydrobromide, and organic acid salts such as acetate, fumarate, oxalate, citrate, methanesulfonate, benzenesulfonate, tosylate or maleate. In addition, in case of compound having an acidic group such as carboxy, salts with a base (for example, alkali metal salts such as a sodium salt and a potassium salt, alkaline earth metal salts such as a calcium salt, organic base salts such as a triethylamine salt, or amino acid salts such as a lysine salt) can be mentioned.

The bicyclic compound (1) or the pharmaceutically acceptable salt thereof includes any of its internal salts, and solvates such as hydrates.

In the bicyclic compound (1) of the present invention, an optical isomer based on an asymmetric carbon may be present, and any of the isomers and a mixture thereof may be encompassed in the bicyclic compound (1) of the present invention. In addition, cis form and trans form may be present, in case that the bicyclic compound (1) has a double bond or a cycloalkanediyl moiety, and a tautomer may be present based on an unsaturated bond such as carbonyl in the bicyclic compound (1), and any of these isomers and a mixture thereof may be encompassed in the bicyclic compound (1).

The bicyclic compound (1) can be prepared as the following methods.
Method 1 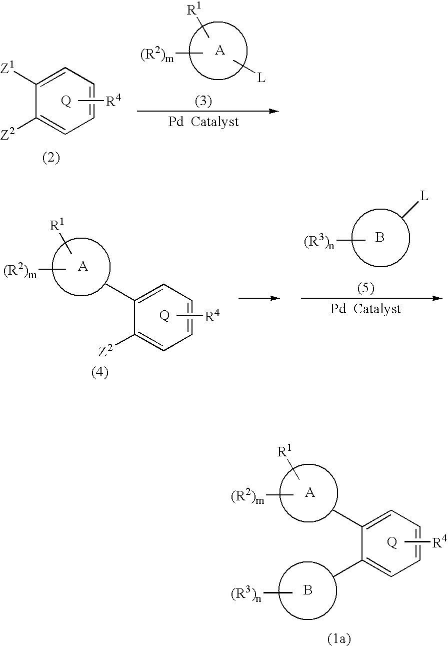
wherein Z¹ is chlorine, bromine, an alkylsulfonyloxy or trifluoromethanesulfonyloxy, Z² is hydroxyl or amino, each of which may have a protective group, L is —B(OH)₂, —B(OR)₂ or —Sn(R)₃, R is an alkyl, and other symbols have the same meanings as defined above.

This method can be carried out by referring to Bioorg. Med. Chem. Lett., 1998, 8, 2777 and WO 98/03484.

The compound (4) can be synthesized by reacting the compound (2) with the compound (3) in the presence of a palladium catalyst. The palladium catalyst may be exemplified by a zero-valent or di-valent palladium catalyst such as tetrakis(triphenylphosphine) palladium (0), bis(triphenylphosphine) palladium (II) chloride, palladium (II) acetate, etc. When the reaction is carried out by using the compound (3) where L is —B(OH)₂ or —B(OR)₂, a base is preferably presented. The base may be exemplified by inorganic bases such as an alkali metal carbonate, an alkali metal hydroxide, an alkali metal phosphate, an alkali metal fluoride, etc., and organic bases such as triethylamine, etc. The solvent is not specifically limited so long as it does not exert any bad effect on the reaction, and may be exemplified by dimethoxyethane (DME), tetrahydrofuran (THF), dioxane, dimethylformamide (DMF), dimethylacetamide (DMA), toluene, benzene or a mixture thereof. The reaction proceeds generally at 60 to 150° C., preferably 80 to 120° C., and for generally from 1 to 24 hours.

The compound (1a) can be prepared by converting Z² of the compound (4) into Z¹ according to the conventional manner, and then, the resulting compound is reacted with the compound (5) in the presence of a palladium catalyst in the same manner.

Incidentally, the bicyclic compound (1a) can be suitably prepared by firstly reacting the compound (2) with the compound (5), and after converting Z² by the same manner, reacting with the compound (3), or a compound in which different kinds of two halogens are introduced may be used as described in Example 20 below.
Method 2 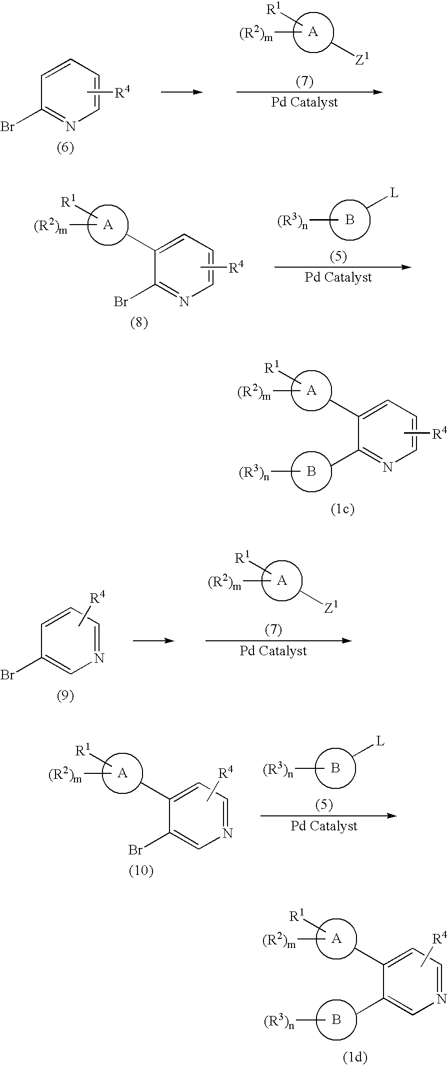
wherein the symbols have the same meanings as defined above.

This method can be carried out by referring to Org. Lett., 2001, 3, 835. Incidentally, explanation is now made by referring to the pyridines (1c) and (1d) in which the nitrogen of the pyridine is positioned in the above chemical formula, but positional isomers of the bicyclic compound can be prepared in the similar manner by changing the order of introduction of Ring A and Ring B.

The compound (8) or (10) can be prepared by treating the compound (6) or the compound (9) with an organic lithium reagent (lithium diisopropylamide, etc.) to prepare a pyridyllithium, subsequently reacting with zinc chloride to prepare a pyridylzinc, and reacting with the compound (7) in the presence of a palladium catalyst in the same manner as in Method 1. The reaction solvent at the time of converting into the pyridyllithium and the pyridylzinc is not specifically limited so long as it does not exert any bad effect on the reaction, and may be exemplified by dimethoxyethane, THF, dioxane, toluene, benzene or a mixture thereof. The reaction proceeds generally at −110 to −50° C. Preparation of the compound (1c) or (1d) from the compound (8) or (10) can be carried out in the same manner as in Method 1.

Method 3

The compound (1e) represented by the following formula can be prepared in the same manner as in Method 1, and can be also prepared as follows. 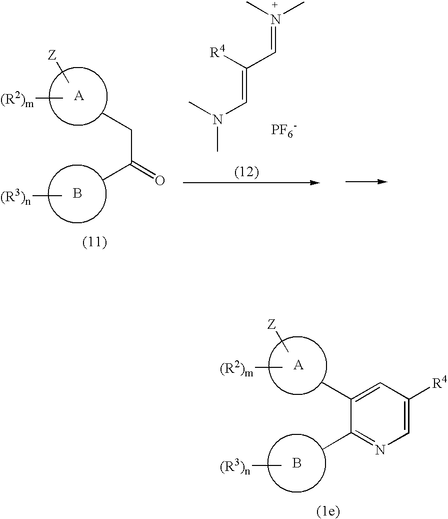
wherein the respective symbols have the same meanings as defined above.

The pyridine (1e) which is substituted by R⁴ at the β-position can be prepared by referring to Org. Lett., 2000, 15, p. 2339, and the compound (11) and compound (12) which are starting material thereof can be prepared by referring to J. Org. Chem., 2000, 65, p. 8415 and J. Org. Chem., 2000, 65, p. 4571, respectively. Incidentally, explanation is now made by referring to the pyridine (1e) in which the nitrogen of the pyridine is positioned in the above chemical formula, but a pyridine substituted by Ring A at the 2-position and by Ring B at 3-position can be similarly prepared.

The ketone (11) is treated with an alkali alkoxide such as potassium tert-butoxide, etc. in an ether such as THF, diethyl ether, ethyleneglycol dimethyl ether, dioxane, etc., at a temperature of 0° C. to 40° C., then, reacted with the compound (12), the resulting reaction mixture is added dropwise to a mixed acid of acetic acid and trifluoroacetic acid, and finally subjecting to ring closure with ammonia such as aqueous ammonia, etc. at a temperature of from 50° C. to the boiling point of the solvent to prepare the compound (1e).
Method 4 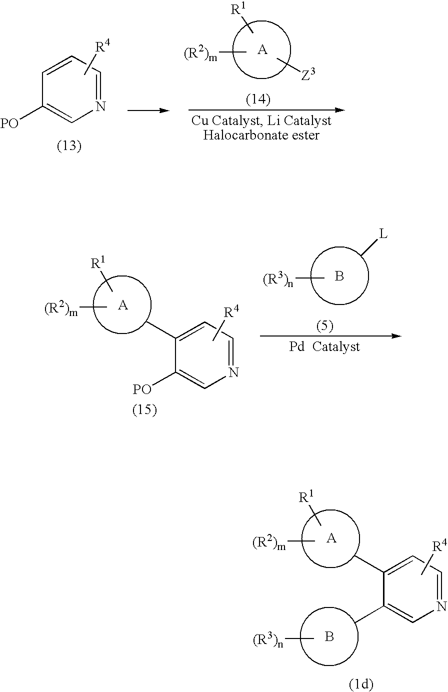
wherein Z³ is Zn-Z or Mg-Z, Z is chlorine, bromine or iodine, P is a protective group for hydroxyl (benzyl, etc.), and other symbols have the same meanings as defined above.

The compound (15) can be prepared from the compound (13) by referring to J. Org. Chem., 62, 503 (1997), and subsequently the compound (1d) can be prepared from the compound (15) in the same manner as in Method 1.

Incidentally, explanation is now made by referring to the pyridine in which the nitrogen of the pyridine is positioned in the above chemical formula, but a pyridine derivative substituted by Ring A at the 3-position and by Ring B at the 4-position can be similarly prepared.
Method 5 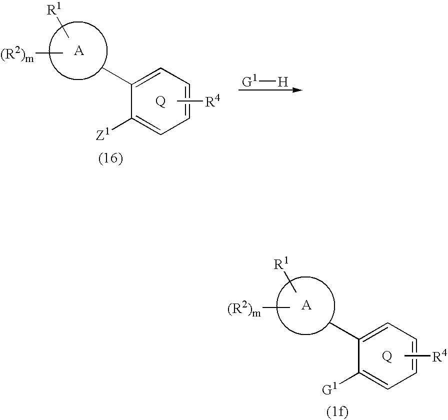
wherein G¹ is an optionally substituted amino, and other symbols have the same meanings as defined above.

The compound in which G¹ in the formula (1) is an amino can be prepared according to Method 1. Also, the compound (1f) in which G¹ is a substituted amino can be prepared by reacting the compound (16) which can be prepared in accordance with Method 1 with an amine G¹-H.

When the substituent Z¹ is positioned adjacent to N, it can be prepared by referring to Example 83. The solvent is not specifically limited so long as it does not exert any bad effect on the reaction, and may be exemplified by dichloromethane, chloroform, THF, dioxane, DMF, DMA, toluene or a mixture thereof. The reaction proceeds generally at 0° C. to 150° C., preferably at room temperature to the boiling point of the used solvent. The reaction time is generally 1 hour to 3 days. Incidentally, the reaction may be optionally carried out in the presence of a base. The base may be exemplified by an inorganic base such as an alkali metal carbonate, and an organic base such as triethylamine.

When the substituent Z¹ is positioned not adjacent to N, such a compound can be prepared by amination reaction using a palladium catalyst according to the method as disclosed in Acc. Chem. Res. 31 (1998), 805 or Angew. Chem., Int. Ed. 37 (1998), 2046.

Method 6

The carboxylic acid (1i) can be prepared from the compound (1g) included in the compound (1) which can be prepared by the above-mentioned Methods and Example as follows. 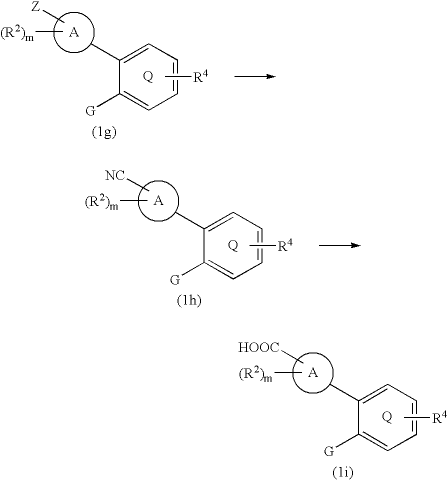
wherein the respective symbols have the same meanings as defined above.

The compound (1g) is reacted with a cyanating reagent (sodium cyanide, cuprous cyanide, etc.) in a solvent (acetonitrile, dimethylsulfoxide, DMF, a mixture thereof, etc.) at room temperature to 100° C. for 1 to 24 hours to synthesize the nitrile (1h). Also, it can be also prepared by reacting with a cyanating reagent such as zinc cyanide, potassium cyanide, etc. in the presence of a tetrakis(triphenylphosphine)palladium catalyst, etc.

The nitrile (1 h) is hydrolyzed by using an acid (hydrochloric acid, sulfuric acid, etc.) or a base (sodium hydroxide, potassium hydroxide, etc.) in a solvent (water, methanol, ethanol, isopropyl alcohol, tert-butyl alcohol, ethylene glycol, diethylene glycol, a mixture thereof, etc.) to give the carboxylic acid (1i). The reaction proceeds generally at −20 to 150° C. for generally 30 minutes to 48 hours.

Also, the compound (1 h) can be prepared according to the same manner as in Method 1.

Incidentally, the nitrile (1 h) is hydrolyzed by using an alkali hydroxide (sodium hydroxide, potassium hydroxide, etc.) in a solvent (water, methanol, ethanol, isopropyl alcohol, tert-butyl alcohol, ethylene glycol, diethylene glycol, a mixture thereof, etc.) to directly give the compound (1j)−1 where R⁵ and R⁶ are both hydrogens.

Method 7

The carboxylic acid (1i) is reacted with a corresponding compound according to the conventional manner to give the following bicyclic compounds (1j) to (1s).

More specifically, for example, it can be carried out as follows. 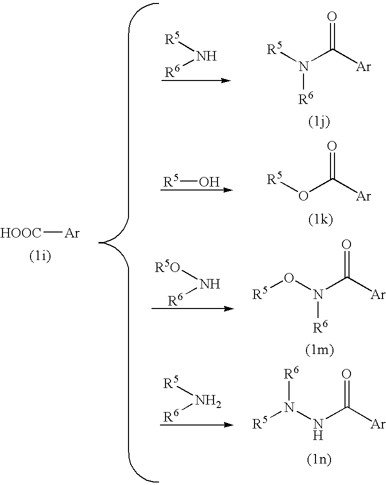
wherein Ar is a residue of the compound (1i), and other symbols have the same meanings as defined above.

The compounds (1j), (1k), (1m) and (1n) included in the bicyclic compound (1) can be prepared by any of the following methods.

(A) The carboxylic acid (1i) is converted into an acid halide by treating the same with a halogenating agent (thionyl chloride, etc.). Then, it is reacted with respective reagents as shown in the reaction formulae, in the presence of a base (sodium bicarbonate, potassium carbonate, triethylamine, pyridine, etc.) at −78° C. to room temperature for 30 minutes to 24 hours to give the compounds (1j), (1k), (1m) and (1n).

(B) The carboxylic acid (1i) is treated with respective reagents as shown in the reaction formulae, in a solvent (DMF, THF, dioxane, etc.), in the presence of a condensing agent (1,3-dicyclohexylcarbodiimide, 1-ethyl-3-(3-dimethylaminopropyl)carbodiimide, carbonyldiimidazole, diethyl cyanophosphate, etc.), to give the compounds (1j), (1k), (1m) and (1n). The reaction proceeds generally at 0° C. to 100° C., and for generally from 30 minutes to 24 hours. In the reaction using the condensing agent, it can be optionally carried out in the presence of 1-hydroxybenztriazole, N-hydroxysuccinimide, etc.
(C) The carboxylic acid (1i) is converted into a mixed anhydride with a monoalkyl carbonate such as methyl carbonate and isobutyl carbonate, or a mixed acid anhydride with an organic acid such as pivalic acid and isovaleric acid, which is then reacted with respective reagent as shown in the reaction formulae, in a suitable solvent (THF, toluene, nitrobenzene, a mixture thereof, etc.), in the presence of a base (triethylamine, pyridine, etc.), at −20° C. to room temperature for 1 to 24 hours to give the compounds (1j), (1k), (1m) and (1n). 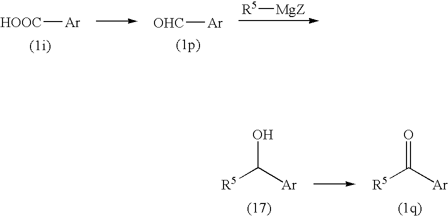
wherein the respective symbols have the same meanings as defined above.

The compound (1q) included in the bicyclic compound (1) can be prepared by the following methods. An aldehyde (1p) prepared from the carboxylic acid (1i) by a conventional method is reacted with a Grignard reagent in a solvent (THF, diethyl ether, ethyleneglycol dimethyl ether, benzene, toluene, xylene, dioxane, etc.) at −20 to 100° C. for 30 minutes to 24 hours to give an alcohol (17). Then, the alcohol (17) is reacted with an oxidizing agent at −78 to 100° C. for 30 minutes to 24 hours to give the compound (1q). As the oxidizing agent, there may be used chromic acid-sulfuric acid, chromium (VI) oxide-sulfuric acidacetone (Jones reagent), chromium (VI) oxide-pyridine complex (Collins reagent), dichromate (such as sodium dichromate, potassium dichromate, etc.)—sulfuric acid, pyridinium chlorochromate (PCC), manganese dioxide, dimethyl sulfoxide-electrophilic activating agent (such as dicyclohexylcarbodiimide, acetic anhydride, phosphorus pentaoxide, a sulfur trioxide-pyridine complex, trifluoroacetic anhydride, oxalyl chloride, and halogen), sodium hypochlorite, potassium hypochlorite, sodium bromite, etc. 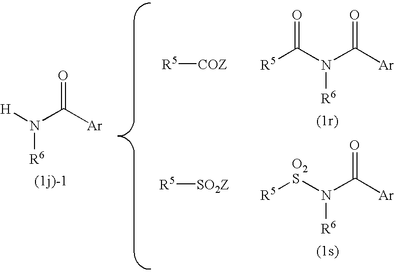
wherein the respective symbols have the same meanings as defined above.

The compounds (1r) and (1s) included in the bicyclic compound (1) can be prepared by the following methods. The compound (1j)−1 included in the compound (1j) is reacted with an acid halide as shown in the above reaction formulae, in the presence of a base (sodium bicarbonate, potassium carbonate, triethylamine, pyridine, etc.) at −20° C. to room temperature for 30 minutes to 24 hours to give the compounds (1r) and (1s).

Incidentally, in the above-mentioned methods, when the bicyclic compound of the present invention, an intermediate compound, a starting compound, etc. have a functional group (hydroxyl, amino, carboxy, etc.), the reaction can be carried out by protecting the functional group with a protective group generally used in an organic synthesis chemistry, and after the reaction, the protective group is removed to give the objective compound. The protective group for hydroxyl may include tetrahydropyranyl, trimethylsilyl, benzyl, etc., the protective group for amino may include tert-butoxycarbonyl, benzyloxycarbonyl, etc., and the protective group for carboxy may include an alkyl such as methyl, ethyl, etc., benzyl, and the like.

Further, after the bicyclic compound of the present invention and the intermediate compound are prepared according to the above-mentioned methods, the functional group can be converted or modified according to the conventional method. Specifically, the following methods are mentioned.

(1) Modification of Amino

After an amino is optionally protected, (i) a reaction with an alkyl halide, etc. may be carried out in the presence of a base (sodium hydride, triethylamine, sodium carbonate, potassium carbonate, etc.), or (ii) an alcohol, etc. may be subjected to Mitsunobu Reaction using dialkyl azodicarboxylate and triphenylphosphine, and deprotection may be optionally carried out to convert the amino to a mono- or di-alkylamino.

(2) Conversion of Amino to Amide

An amino may be converted to a corresponding amide by reacting with an acyl halide.

(3) Conversion of Carboxy to Carbamoyl

Carboxy may be converted to a corresponding carbamoyl by racting with an amine.

(4) Hydrogenation of C═C Double Bond

A C═C double bond may be converted to a corresponding single bond by catalytic reduction using a transition metal (platinum, palladium, rhodium, ruthenium, nickel, etc.) catalyst.

(5) Hydrolysis of Ester

An ester may be converted to a corresponding carboxy by hydrolysis using an alkali (sodium hydroxide, potassium hydroxide, etc.).

(6) Conversion of Carbamoyl to Nitrile

Carbamoyl may be converted to a corresponding nitrile by reacting with trifluoroacetic anhydride.

(7) Conversion of Carboxy to 4,5-dihydroxazol-2-yl

Carboxy may be converted to a corresponding 4,5-dihydroxazol-2-yl by reacting with 2-haloethylamine in the presence of a condensing agent.

(8) Halogenation and Alkylation of Hydroxyl

Hydroxyl may be converted to a corresponding halide by reacting with a halogenating agent. Also, the halide may be converted to a corresponding alkoxy by reacting with an alcohol.

(9) Reduction of Ester

Ester may be converted to a corresponding hydroxyl by reduction using a reducing agent (a metal reducing agent such as lithium aluminum hydride, sodium borohydride, lithium borohydride, etc., diborane, etc.).

(10) Oxidation of Hydroxyl

Hydroxyl may be converted to an aldehyde, ketone or carboxy by oxidation.

(11) Amination of Ketone, Aldehyde

Ketone or aldehyde may be converted to a mono- or di-substituted aminomethyl by reductive amination with an amine in the presence of a reducing agent (sodium borohydride, sodium cyanoborohydride, etc.).

(12) Conversion of Ketone or Aldehyde to Double Bond

Ketone or aldehyde may be converted to a double bond by Wittig reaction.

(13) Conversion of Sulfonamide to Salt

Sulfonamide may be converted to a corresponding sulfonamide salt (a sodium salt, a potassium salt, etc.) by treating with sodium hydroxide, potassium hydroxide, etc. in an alcohol (methanol, ethanol, etc.).

(14) Conversion of Aldehyde to Oxime, Etc.

Aldehyde may be converted to a corresponding oxime by reacting with hydroxylamine or O-alkylhydroxylamine in the presence of a base (sodium bicarbonate, etc.) in an alcohol (methanol, ethanol, etc.).

(15) Conversion of Halide to Nitrile

Halide may be converted to a corresponding nitrile by reacting with a cyanating agent.

(16) Amination of Halide

A halide may be converted to a corresponding amine according to the method disclosed in Tetrahedron, 2002, p. 2041.

(17) Conversion of Carboxylic Acid to Carbamoyl or Hydroxymethyl

Carboxylic acid may be converted to a corresponding carbamoyl by condensating with N-hydroxysuccinimide to give succinimide ester, and reacting with an amine. Also, the succinimide ester may be converted to a corresponding hydroxymethyl by treating with a reducing agent (sodium borohydride, etc.).

(18) Dehalogenation

A halogen-substituted aromatic ring may be dehalogenated by catalytic reduction. Also, it can be dehalogenated by reacting with potassium methoxide in the presence of a palladium catalyst according to the method disclosed in Organometallics 2001, 20, 3607 and Example 4.

(19) Conversion of Aryl Halide

A halide may be converted to a corresponding amino, alkoxy or aryloxy by reacting an aryl halide or heteroaryl halide with a nucleophilic reagent (a primary amine, a secondary amine, an alcohol, phenol, etc.) according to Method 5.

(20) Alkylation of Heteroaryl Halide

A halogen may be converted to an alkyl according to the method disclosed in Chem. Commun., 1996, 2719, J. Chem. Soc., Chem. Commun., 1988, 638, or Tetrahedron Lett., 37, 1309 (1996).

In the above-mentioned preparation methods, each of the prepared compounds and intermediates may be purified by a conventional method such as column chromatography, recrystallization, etc. Examples of the recrystallization solvent include an alcohol solvent such as methanol, ethanol, 2-propanol, etc., an ether solvent such as diethyl ether, etc., an ester solvent such as ethyl acetate, etc., an aromatic solvent such as toluene, etc., a ketone solvent such as acetone, a hydrocarbon solvent such as hexane, etc., water, and a mixed solvent thereof. The bicyclic compound of the present invention can be converted to a pharmaceutically acceptable salt according to the conventional method, and subsequently subjected to recrystallization, etc.

The bicyclic compound (1) or a pharmaceutically acceptable salt thereof may be prepared into a pharmaceutical composition comprising a therapeutically effective amount of the compound and a pharmaceutically acceptable carrier. The pharmaceutically acceptable carrier may include a diluent, a binder (such as syrup, Gum Arabic, gelatin, sorbit, tragacanth and polyvinyl pyrrolidone), an excipient (such as lactose, sucrose, corn starch, potassium phosphate, sorbit and glycine), a lubricant (such as magnesium stearate, talc, polyethylene glycol and silica), a disintegrator (such as potato starch) and a humectant (such as lauryl sodium sulfate).

The bicyclic compound (1) or a pharmaceutically acceptable salt thereof can be administered orally or parenterally, and used as suitable pharmaceutical preparations. The pharmaceutical preparation for oral administration may include solid preparations such as tablets, granules, capsules, and powders, or liquid preparations such as solutions, suspensions and emulsions. The pharmaceutical preparation for parenteral administration may include a suppository, an injection or a drip infusion by using distilled water for injection, physiological saline or an aqueous glucose solution, or an inhalant, etc.

A dose of the bicyclic compound (1) or a pharmaceutically acceptable salt thereof may vary depending on an administration route, an age, body weight or conditions of a patient, or a kind or degree of a disease, and generally about 0.1 to 50 mg/kg per day, more preferably about 0.1 to 30 mg/kg per day.

### EFFECTS OF THE INVENTION

The bicyclic compound (1) of the present invention or a pharmaceutically acceptable salt thereof has an excellent large conductance calcium-activated K channel opening activity and hyperpolarizes a membrane electric potential of cells, so that it may be used for a prophylactic, relief and/or treatment agent of, for example, hypertension, premature birth, irritable bowel syndrome, chronic heart failure, angina, cardiac infarction, cerebral infarction, subarachnoid hemorrhage, cerebral vasospasm, cerebral hypoxia, peripheral blood vessel disorder, anxiety, male-pattern baldness, erectile dysfunction, diabetes, diabetic peripheral nerve disorder, other diabetic complication, sterility, urolithiasis and pain accompanied thereby, pollakiuria, urinary incontinence, nocturnal enuresis, asthma, chronic obstructive pulmonary diseases (COPD), cough accompanied by asthma or COPD, cerebral apoplexy, cerebral ischemia, traumatic encephalopathy, and the like.

### BEST MODE FOR CARRYING OUT THE INVENTION

In the following, the present invention will be explained in detail by referring to Examples and Reference examples, but the present invention is not limited by these.

### EXAMPLES

### Example 1

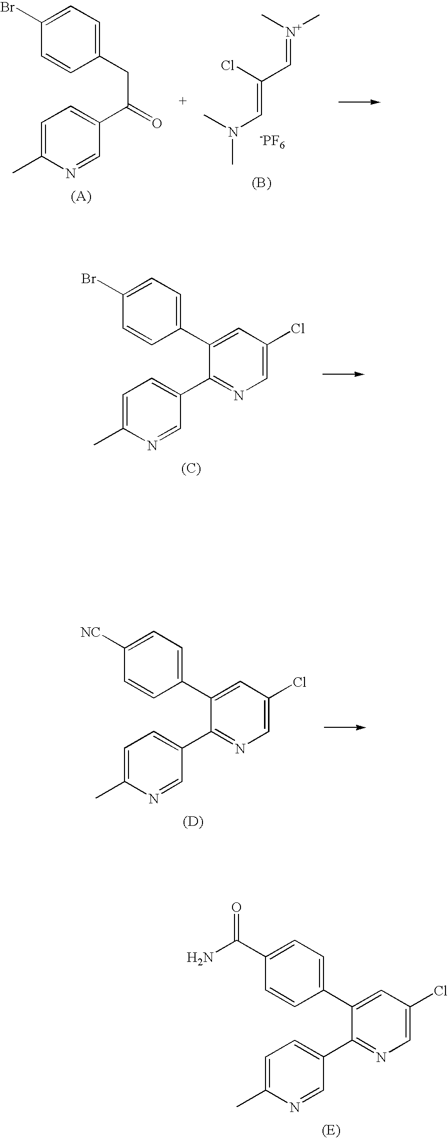 (1) Compound A (12.0 g, 41.4 mmol) was dissolved in THF (200 ml), and a solution of potassium tert-butoxide (6.09 g, 54.3 mmol) in THF (50 ml) was added dropwise to the solution at 0° C. under argon atmosphere over a period of 30 minutes. After completion of addition, the mixture was stirred at room temperature for one hour, and then, Compound B (12.7 g, 41.4 mmol) was added thereto at once. The mixture was stirred for 45 minutes, and the reaction mixture was added dropwise through cannula to a mixture of trifluoroacetic acid (3.19 ml, 41.4 mmol) and acetic acid (20.7 ml, 362 mmol) under argon atmosphere at room temperature. The mixture was stirred at room temperature for one hour, 28% aqueous ammonia (250 ml) was added to the mixture, and the resulting mixture was refluxed overnight. The reaction mixture was cooled down to room temperature, and concentrated under reduced pressure to about a half volume. The concentrate was extracted with ethyl acetate (400 ml), and the extract was washed with water, dried over anhydrous magnesium sulfate and concentrated. The residue was purified by NH silica gel column chromatography (hexane:ethyl acetate=9:1→1:1) to give Compound C (12.42 g, 88%) as a solid.
MS: 359/361 [M+H]⁺, APCI (MeOH)
(2) A suspension of Compound C (4.00 g, 11.1 mmol), zinc cyanide (1.306 g, 11.1 mmol), tetrakis(triphenylphosphine)palladium (1.285 g, 1.1 mmol) in DMF (50 ml) was heated to 80° C. and stirred for one hour. The suspension was poured into ethyl acetate/water, and the organic layer was washed with brine, dried over anhydrous sodium sulfate, and concentrated under reduced pressure. The residue was purified by silica gel column chromatography (hexane:ethyl acetate=6:1) to give Compound D (2.79 g, 82%) as powders. MS: 306/308 [M+H]⁺, APCI (MeOH)
(3) To a solution of Compound D (100 mg, 0.327 mmol) in tert-butanol (5.0 ml) was added powdered potassium hydroxide (165 mg, 2.94 mmol), and the mixture was refluxed under stirring for 2 hours. The reaction mixture was cooled down, brine was added thereto, and the resulting mixture was extracted with ethyl acetate. The organic layer was dried over anhydrous sodium sulfate, and concentrated under reduced pressure. The residue was purified by silica gel column chromatography (chloroform:methanol=100:0→90:10) to give Compound E (102 mg, 96%) as a solid.
MS: 324/326 [M+H]⁺, APCI (MeOH)

### Example 2

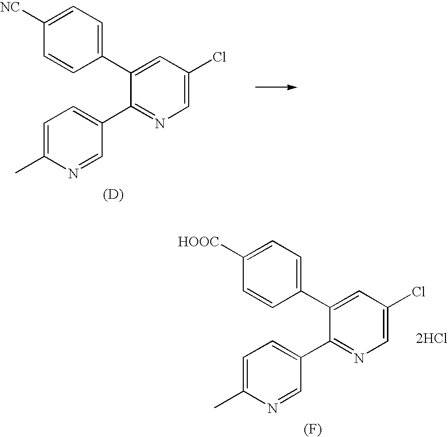

To Compound D (2.69 g, 8.80 mmol) prepared in Example 1 was added 6N hydrochloric acid (50 ml), and the mixture was refluxed for 4 hours under stirring. The reaction mixture was concentrated, and the residue was triturated with ethanol-ether to give Compound F (3.34 g, 100%) as a solid.

MS: 323/325 [M-H]⁻, ESI (MeOH)

### Example 3

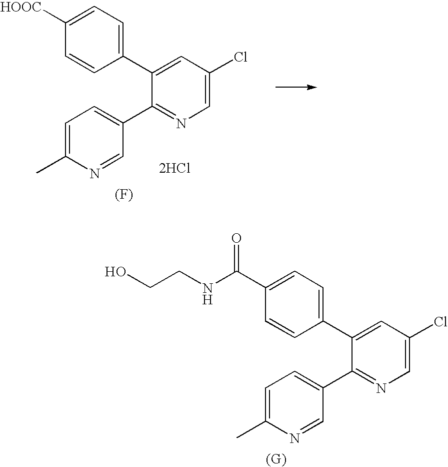

To Compound F (76 mg, 0.20 mmol) prepared in Example 2 was added thionyl chloride (0.5 ml), and the mixture was refluxed for one hour under stirring. The reaction mixture was concentrated, suspended in dichloromethane, and added to an ice-cooled solution of ethanolamine (24 mg, 0.40 mmol) and triethylamine (112 μl, 0.80 mmol) in dichloromethane (2 ml). The mixture was stirred at room temperature overnight, and the reaction mixture was concentrated. Ethyl acetate was added to the mixture, and the organic layer was washed with water, and concentrated under reduced pressure. The residue was purified by high performance liquid chromatography (methanol-water) to give Compound G (25.2 mg, 34%) as a solid.

MS: 368/370 [M+H]⁺, ESI (MeOH)

### Example 4

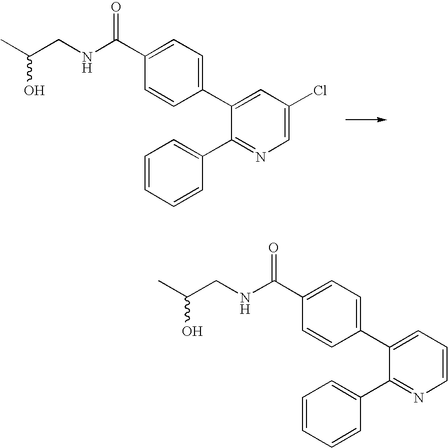

(Compound of Example 11)

The compound (60 mg, 0.15 mmol) of below-mentioned Example 11, (dibenzylideneacetone)palladium (9 mg, 16 μmol), 1,3-bis(2,4,6-trimethylphenyl)imidazolium chloride (5 mg, 15 μmol) and potassium methoxide (21 mg, 0.30 mmol) were suspended in dioxane (10 ml), and the suspension was stirred at 100° C. under argon atmosphere for 2 hours. After removing insolubles by filtration, the reaction mixture was concentrated, and the residue was purified by silica gel column chromatography (chloroform:methanol=90:10) to give N-(2-hydroxypropyl)-4-(2-phenylpyridine-3-yl)benzamide (3.6 mg, 7%) as a solid.

MS: 333 [M+H]⁺, APCI (MeOH)

### Examples 5 to 8

The following compounds were prepared by carrying out reactions in the same manner as in Examples 1 and 2.

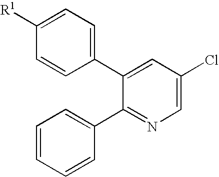

| Example | R¹ | Salt | MS |
|---|---|---|---|
| Example 5 | Br— | | 344/346 [M + H]⁺, APCI (MeOH) |
| Example 6 | NC— | | 291/293 [M + H]⁺, APCI (MeOH) |
| Example 7 | HOOC— | | 308/310 [M − H]⁺, ESI (MeOH) |
| Example 8 | H₂NCO— | HCl | 309/311 [M + H]⁺, APCI (MeOH) |

### Examples 9 to 19

The following compounds were prepared by carrying out reactions in the same manner as in Example 3.

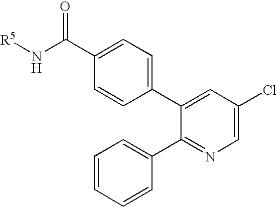

| Example | R⁵ | Salt | MS |
|---|---|---|---|
| Example 9 | HO—(CH₂)₃— | HCl | 367/369 [M + H]⁺, |
| | | | APCI (MeOH) |
| Example 10 | HO—(CH₂)₂— | HCl | 353/355 [M + H]⁺, |
| | | | APCI (MeOH) |
| Example 11 | CH₃CH(OH)CH₂— | HCl | 367/369 [M + H]⁺, |
| | | | APCI (MeOH) |
| Example 12 | CH₃OCONH—(CH₂)₂— | HCl | 410/412 [M + H]⁺, |
| | | | APCI (MeOH) |
| Example 13 | HOCH₂CH(OH)CH₂— | HCl | 383/385 [M + H]⁺, |
| | | | APCI (MeOH) |
| Example 14 | (2R)-HOCH₂CH(OH)CH₂— | | 383/385 [M + H]⁺, |
| | | | APCI (MeOH) |
| Example 15 | (2S)-HOCH₂CH(OH)CH₂— | | 383/385 [M + H]⁺, |
| | | | APCI (MeOH) |

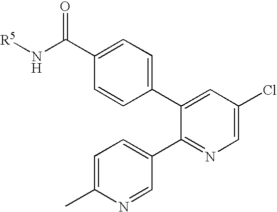

| Example | R⁵ | MS |
|---|---|---|
| Example 16 | HO—(CH₂)₃— | 382/384 [M + H]⁺, ESI (MeOH) |
| Example 17 | CH₃O—(CH₂)₂— | 382/384 [M + H]⁺, ESI (MeOH) |
| Example 18 | CH₃SO₂NH—(CH₂)₂— | 445/447 [M + H]⁺, ESI (MeOH) |

### Example 19

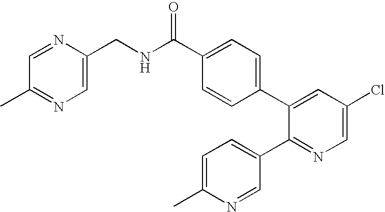 MS: 430/432 [M+H]⁺, ESI (MeOH)

### Example 20

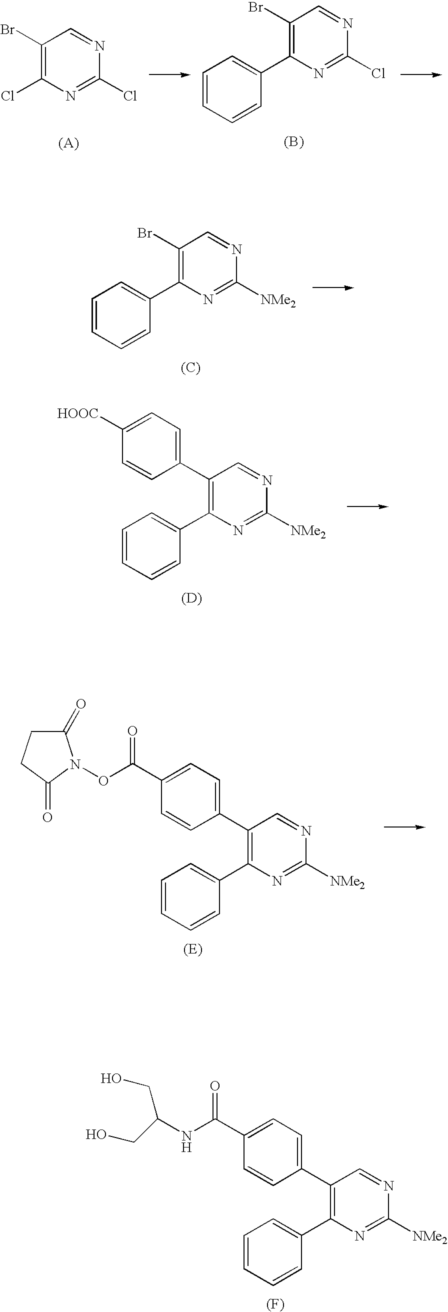 (1) Compound A (10.95 g, 48 mmol: J. Chem. Soc., 1646 (1953)), tri(n-butyl)phenyltin (20.0 g, 0.54 mol), and bis(triphenylphosphine)palladium (II) dichloride (1.01 g, 1.4 mmol) were heated in DMF (100 ml) at 70° C. for 18 hours. After the mixture was cooled down, ethyl acetate and an aqueous 20% potassium fluoride solution were added to the mixture, and the resulting mixture was stirred. After removing the precipitates by filtration, the filtrate was extracted with ethyl acetate. The organic layer was washed with water, dried over anhydrous sodium sulfate, and concentrated under reduced pressure. The residue was purified by silica gel column chromatography (hexane:ethyl acetate=97:3) to give Compound B (6.82 g, 53%) as a solid.
MS: 269/271 [M+H]⁺, APCI
(2) Compound B (0.45 g, 1.67 mmol), dimethylamine hydrochloride (0.54 g, 6.62 mmol), and triethylamine (1.39 ml, 9.97 mmol) were dissolved in ethanol (15 ml), and the mixture was refluxed for 15 hours. The solvent was removed under reduced pressure, and the obtained residue was diluted with water and extracted with ethyl acetate. The organic layer was washed with water, dried over anhydrous sodium sulfate, and concentrated under reduced pressure. The residue was purified by silica gel column chromatography (hexane:ethyl acetate=50:1) to give Compound C (0.38 g, 82%) as a solid.
MS: 278/280 [M+H]⁺, APCI
(3) Compound C (1 g, 3.60 mmol), 4-carboxy phenylboric acid (656 mg, 3.95 mmol), bis(triphenylphosphine)palladium (II) dichloride (252 mg, 0.36 mmol), and an aqueous 2N sodium carbonate solution (7.2 ml) were heated in DME (7.2 ml) at 100° C. under microwave irradiation for one hour. After the mixture was cooled down, water and diethyl ether were added to the reaction mixture. The aqueous layer was obtained by separation, acetic acid was added to the layer until it became a pH of 4, and the aqueous layer was extracted with ethyl acetate. The organic layer was washed with water, dried over anhydrous sodium sulfate, and concentrated under reduced pressure to give crude Compound D as a solid. The obtained Compound D and N-hydroxysuccinimide (0.497 g, 4.32 mmol) were dissolved in DMF (5 ml), and 1-ethyl-3-(3-dimethylaminopropyl)carbodiimide hydrochloride (0.754 g, 3.95 mmol) was added to the solution under ice-cooling, and the resulting mixture was stirred at room temperature for 3 days. The reaction mixture was diluted with water and extracted with ethyl acetate. The organic layer was washed with water, dried over anhydrous sodium sulfate, and concentrated under reduced pressure. The residue was purified by silica gel column chromatography (hexane:ethyl acetate-90:10→1:1) to give Compound E (869 mg, 58%) as a solid.
MS: 417 [M+H]⁺, APCI
(4) Compound E (75 mg, 0.18 mmol), 2-amino-1,3-propanediol (33 mg, 0.36 mmol), and pyridine (0.05 ml) were dissolved in a mixture of THF (5 ml) and DMF (5 ml), and stirred at 50° C. for 3 hours. The reaction mixture was cooled and diluted with water, extracted with ethyl acetate. The organic layer was washed with water, dried over anhydrous sodium sulfate, and concentrated under reduced pressure. The residue was purified by silica gel column chromatography (chloroform:methanol=100:0˜90:10) to give Compound F (20 mg, 28%) as a solid.
MS: 393 [M+H]⁺, APCI

### Example 21

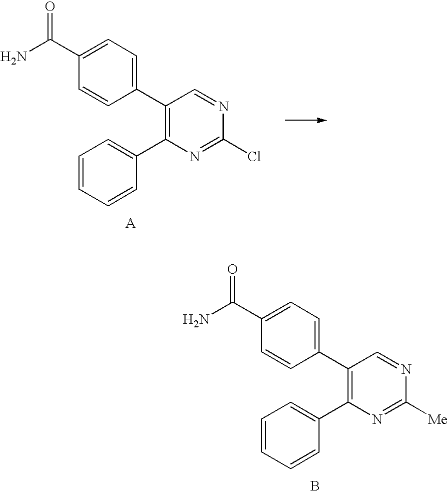

A suspension of Compound A (100 mg, 0.32 mmol) described in Example 124, trimethylaluminum (0.97 ml of 1.0M hexane solution, 0.97 mmol), tetrakis(triphenylphosphine) palladium (74 mg, 64 μmol) in dioxane (3 ml) was stirred under argon atmosphere at 70° C. for 9 hours. After the reaction mixture was cooled to 0° C., an aqueous saturated K₂CO₃ solution was added to the mixture. The mixture was extracted with ethyl acetate, dried over anhydrous sodium sulfate, and concentrated under reduced pressure. The residue was purified by silica gel column chromatography (chloroform:methanol=95:5→88:12) to give Compound B (64 mg, 0.22 mmol, 68%) as powders.

MS: 290 [M+H]⁺, APCI (MeOH)

### Example 22

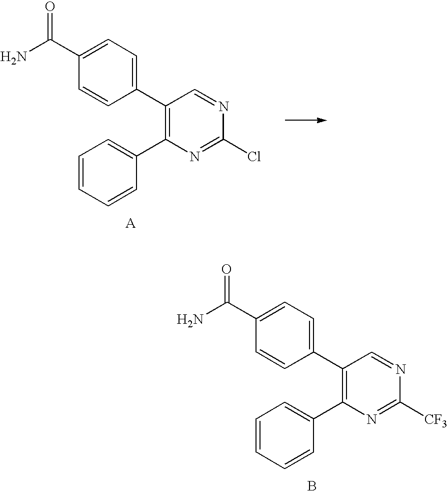

To a suspension of Compound A (500 mg, 1.61 mmol) in dichloromethane (3 ml) was added a conc. aqueous hydrogen iodide solution (3 ml, 12.9 mmol) under ice-cooling, and the resulting mixture was stirred at the same temperature for 8 hours. After the mixture was neutralized by K₂CO₃, iodine was reduced by an aqueous 10% NaHSO₃ solution. Insolubles were collected by filtration, and washed with water and hexane to give crude 2-iodopyrimidine (225 mg). The obtained crude 2-iodopyrimidine was used in the next reaction without purification. A suspension of crude 2-iodopyrimidine (175 mg), copper powder (333 mg, 5.24 mmol), and dibromodifluorocarbon (0.16 ml, 1.75 mmol) in DMA (6 ml) was stirred at 100° C. under argon atmosphere for 6 hours. After the reaction mixture was cooled down, it was diluted with ethyl acetate and washed with water. The organic layer was dried over anhydrous sodium sulfate, and concentrated under reduced pressure. The residue was purified by high performance liquid chromatography (methanol-water) to give Compound B (9 mg, 2%) as powders. MS: 344 [M+H]⁺, APCI (MeOH)

### Examples 23 to 66

The following compounds were obtained according to the methods described in the present specification and according to the methods disclosed in the conventionally known literatures.

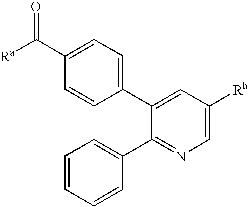

| Example | R^{a} | R^{b} | Salt | MS |
|---|---|---|---|---|
| 23 | HO(CH₂)₂O(CH₂)₂NH— | Cl | HCl | 397/399 |
| | | | | [M + H]⁺, |
| | | | | APCI |
| 24 | Me₂N(CH₂)₂NH— | Cl | 2HCl | 380/382 |
| | | | | [M + H]⁺, |
| | | | | APCI |
| 25 | 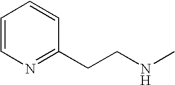 | Cl | 2HCl | 414/416 [M + H]⁺, APCI |
| 26 | (2R)-CH₃CH(OH)CH₂NH— | Cl | HCl | 367/369 |
| | | | | [M + H]⁺, |
| | | | | APCI |
| 27 | (2R)-CH₃CH(OH)CH₂NH— | H | HCl | 333 |
| | | | | [M + H]⁺, |
| | | | | APCI |
| 28 | NH₂— | H | — | 275 |
| | | | | [M + H]⁺, |
| | | | | APCI |
| 29 | NH₂— | CH₃ | HCl | 289 |
| | | | | [M + H]⁺, |
| | | | | APCI |

### Example 30

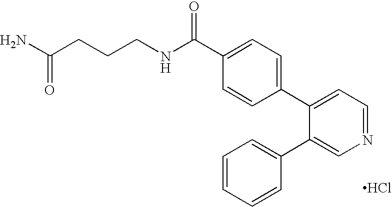 MS: 360 [M+H]⁺, APCI

### Example 31

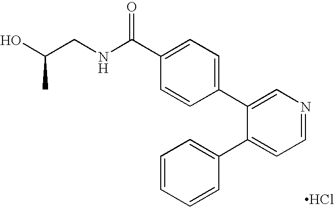

MS: 333 [M+H]⁺, APCI

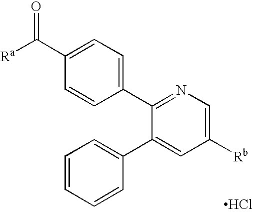

| Example | R^{a} | R^{b} | MS |
|---|---|---|---|
| 32 | 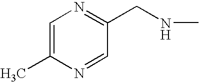 | H | 381 [M + H]⁺, APCI |
| 33 | (2S)-HOCH₂CH(OH)CH₂NH— | Cl | 383/385 [M + H]⁺, |
| | | | APCI |
| 34 | 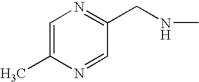 | Cl | 415/417 [M + H]⁺, APCI |

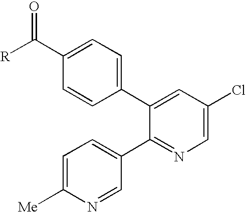

| Example | R | MS |
|---|---|---|
| 35 | 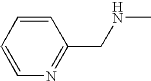 | 415 [M + H]⁺, ESI |
| 36 | 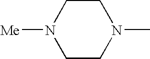 | 407 [M + H]⁺, ESI |
| 37 | 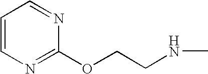 | 446 [M + H]⁺, ESI |
| 38 | 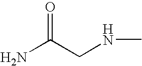 | 381/383 [M + H]⁺, APCI |

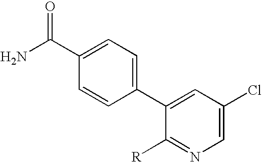

| Exam- | | | |
|---|---|---|---|
| ple | R | Salt | MS |
| 39 | 4-methylphenyl | HCl | 323/325 [M + H]⁺, APCI |
| 40 | 4-fluorophenyl | HCl | 327/329 [M + H]⁺, APCI |
| 41 | 4-methoxyphenyl | HCl | 339/341 [M + H]⁺, APCI |
| 42 | 3-methylphenyl | HCl | 323/325 [M + H]⁺, APCI |
| 43 | 2-methylphenyl | HCl | 323/325 [M + H]⁺, APCI |
| 44 | 4-N,N-dimethylaminophenyl | 2HCl | 352/354 [M + H]⁺, APCI |
| 45 | 1-cyclohexenyl | HCl | 313/315 [M + H]⁺, APCI |
| 46 | 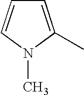 | HCl | 312/314 [M + H]⁺, APCI |
| 47 | 3-pyridyl | HCl | 310/312 [M + H]⁺, APCI |
| 48 | 4-trifluoromethylphenyl | HCl | 377/379 [M + H]⁺, APCI |
| 49 | 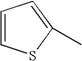 | HCl | 315/317 [M + H]⁺, APCI |
| 50 | 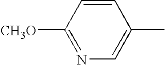 | — | 340/342 [M + H]⁺, APCI |
| 51 | 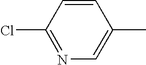 | — | 344/346 [M + H]⁺, APCI |
| 52 | 2-methoxyphenyl | HCl | 339/341 [M + H]⁺, APCI |
| 53 | 3-quinolyl | HCl | 360/362 [M + H]⁺, APCI |

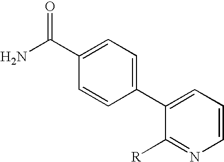

| Example | R | Salt | MS |
|---|---|---|---|
| 54 | 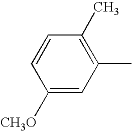 | HCl | 319 [M + H]⁺, APCI |
| 55 | 2-methoxyphenyl | HCl | 305 [M + H]⁺, APCI |
| 56 | 4-trifluorophenyl | HCl | 343 [M + H]⁺, APCI |
| 57 | 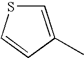 | HCl | 281 [M + H]⁺, APCI |
| 58 | 2-methylphenyl | HCl | 289 [M + H]⁺, APCI |
| 59 | 4-fluorophenyl | HCl | 293 [M + H]⁺, APCI |
| 60 | 4-N,N-dimethylaminophenyl | HCl | 318 [M + H]⁺, APCI |
| 61 | 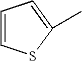 | — | 281 [M + H]⁺, APCI |
| 62 | 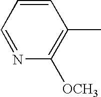 | — | 306 [M + H]⁺, APCI |
| 63 | 1-cyclohexenyl | HCl | 279 [M + H]⁺, APCI |
| 64 | 3-methylphenyl | HCl | 289 [M + H]⁺, APCI |
| 65 | 4-methoxyphenyl | HCl | 305 [M + H]⁺, APCI |
| 66 | 4-methylphenyl | HCl | 289 [M + H]⁺, APCI |

### Example 67

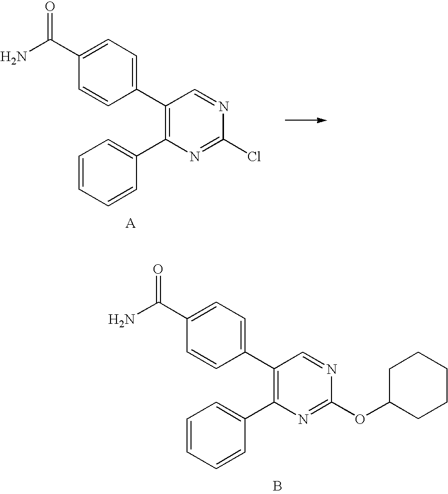

To a suspension of 60% NaH (25 mg, 0.65 mmol) in DMF (3 ml) was added dropwise cyclohexyl alcohol (0.17 ml, 1.6 mmol) under ice-cooling. The reaction mixture was stirred at room temperature for 30 minutes, Compound A (10 mg, 0.32 mmol) was added to the mixture, and stirred at 80° C. for 2.5 hours. After cooling by allowing to stand, water was added to the mixture and the resulting mixture was extracted with ethyl acetate. The organic layer was dried over anhydrous sodium sulfate, and concentrated under reduced pressure. The residue was purified by silica gel column chromatography (hexane:ethyl acetate=3:2→ethyl acetate) to give Compound B (41 mg, 0.11 mmol, 34%) as powders.

MS:374 [M+H]⁺, APCI (MeOH)

### Examples 68 to 82

The following compounds were obtained according to the methods and Examples described in the present specification and the methods disclosed in the conventionally known literatures.

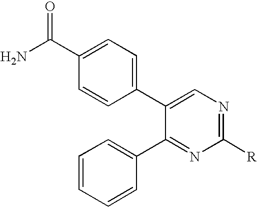

| Example | R | Salt | MS |
|---|---|---|---|
| 68 | H— | — | 276 [M + H]⁺, APCI |
| 69 | (CH₃)₂N— | HCl | 319 [M + H]⁺, APCI |
| 70 | Phenoxy- | — | 368 [M + H]⁺, APCI |
| 71 | C₂H₅O— | — | 320 [M + H]⁺, APCI |
| 72 | 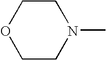 | HCl | 361 [M + H]⁺, APCI |
| 73 | CH₃NH— | HCl | 305 [M + H]⁺, APCI |
| 74 | (CH₃)₂CHO— | — | 334 [M + H]⁺, APCI |
| 75 | (HOCH₂CH₂)₂N— | HCl | 379 [M + H]⁺, APCI |
| 76 | (CH₃OCH₂CH₂)₂N— | HCl | 407 [M + H]⁺, APCI |
| 77 | N-isopropyl-N-methylamino- | HCl | 347 [M + H]⁺, APCI |

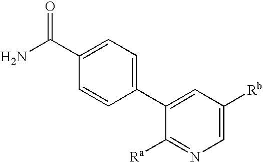

| Example | R^{a} | R^{b} | Salt | MS |
|---|---|---|---|---|
| 78 | NH₂— | Cl | — | 248/250 [M + H]⁺, APCI |
| 79 | 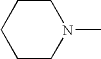 | Cl | HCl | 316/318 [M + H]⁺, APCI |
| 80 | 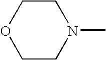 | Cl | HCl | 318/320 [M + H]⁺, APCI |
| 81 | (CH₃)₂CHNH— | Cl | HCl | 290/292 [M + H]⁺, APCI |
| 82 | 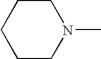 | H | HCl | 282 [M + H]⁺, APCI |

### Example 83

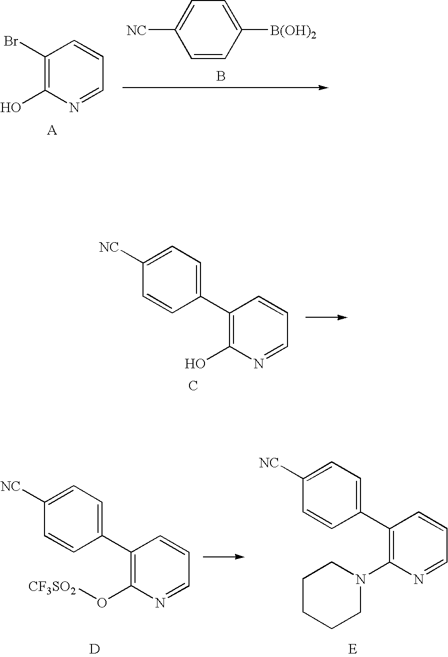 (1) The suspension of Compound A (5.2 g, 29.9 mmol) and Compound B (5.4 g, 35.6 mmol) in DME (50 ml) and 2M aqueous sodium carbonate solution (30 ml) was degassed by ultrasonic wave under reduced pressure to replace the atmosphere with argon. To the mixture was added bis(triphenylphosphine)palladium (II) dichloride (2.1 g, 2.9 mmol), and the resulting mixture was refluxed under argon atmosphere for 16 hours. The reaction mixture was cooled to room temperature, ethyl acetate (300 ml) and water (50 ml) were added to the mixture. The resulting mixture was filtered by using radiolite pad and was extract with etyl acetate. The organic layer was washed with brine, dried by using 10 ml of Chem Elut and concentrated under reduced pressure. The obtained crystalline residue was washed with ethyl acetate to give Compound C (2.65 g, 45%) as a solid.
MS: 197 [M+H]⁺, APCI (MeOH)
(2) To a solution of Compound C (2.65 g, 13.5 mmol) in pyridine (40 ml) was added dropwise trifluoromethane sulfonic anhydride (6.8 ml, 40.4 mmol) under ice-cooling over 10 minutes. A temperature of the mixture was raised to room temperature, and the mixture was stirred for 16 hours, and then, concentrated under reduced pressure. Ethyl acetate and water were added to the residue and were extracted with ethyl acetate. The organic layer was washed with brine, dried by using 5 ml of Chem Elut and concentrated under reduced pressure. The residue was purified by silica gel column chromatography (hexane:ethyl acetate=5:1→3:1) and the obtained crystalline residue was washed with hexane to give Compound D (3.9 g, 88%) as a solid. MS: 329 [M+H]⁺, APCI (MeOH)
(3) The solution of Compound E (98 mg, 0.30 mmol) and piperidine (90 μl, 0.91 mmol) in THF (3 ml) was refluxed for 24 hours, piperidine (90 μl, 0.91 mmol) and THF (3 ml) were additionally added to the solution, and the resulting mixture was refluxed for further 24 hours. The reaction mixture was cooled to room temperature, and concentrated under reduced pressure. The residue was purified by silica gel column chromatography (hexane:ethyl acetate=90:10→70:30) to give Compound E (27.4 mg, 35%) as powders.
MS: 264 [M+H]⁺, APCI (MeOH)

### Example 84

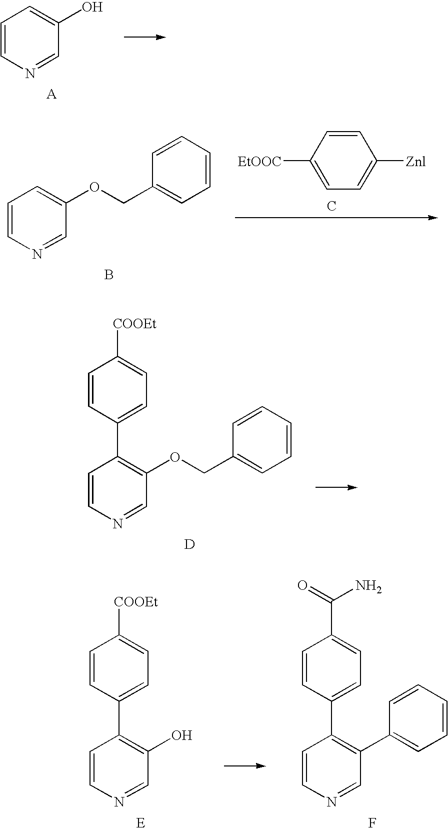 (1) 60% NaH (4.42 g, 111 mmol) was added to a solution of Compound A (10.0 g, 105 mmol) in DMF (100 ml) at 0° C., subsequently benzyl bromide (13.7 ml, 115 mmol) was added to the mixture. Then, the mixture was stirred at room temperature overnight. The reaction mixture was poured into ice-water, and extracted with diethyl ether. The extract was washed with water, dried over anhydrous sodium sulfate, and concentrated under reduced pressure. The residue was purified by silica gel column chromatography (hexane:ethyl acetate=97:350:50) to give Compound B (16.3 g, 84%) as a liquid.
MS: 186 [M+H]⁺, APCI (MeOH)
(2) A solution of phenyl chloroformate (3.45 ml, 27.5 mmol) in THF (20 ml) was added dropwise to a solution of Compound B (4.63 g, 25.0 ml), lithium chloride (0.21 g, 5.0 mmol) and copper (I) iodide (0.476 g, 2.5 mmol) in THF (500 ml) at −23° C. After 20 minutes from completion of the dropwise addition, Compound C (0.5M THF solution, 50 ml, 25.0 mmol) was added dropwise to the mixture at −23° C. After 20 minutes from completion of the dropwise addition, a temperature of the reaction mixture was gradually raised to room temperature. An aqueous 20% ammonium chloride solution (100 ml) and an aqueous 5% ammonia (20 ml) were added to the mixture, and then, THF was removed under reduced pressure. Diethyl ether was added to the residue, and the mixture was washed with water, dried over anhydrous sodium sulfate, and concentrated under reduced pressure. Toluene (200 ml) was added to the residue, and a solution of o-chloranil (6.15 g, 25.0 mmol) in toluene (50 ml) was added dropwise to the mixture at 0° C. After completion of the dropwise addition, the mixture was stirred at room temperature overnight. Diethyl ether was added to the mixture, and the resulting mixture was washed with an aqueous 10% sodium hydroxide solution and water, dried over anhydrous sodium sulfate, and concentrated under reduced pressure. The residue was purified by silica gel column chromatography (hexane:ethyl acetate=95:5→50:50) to give Compound D (2.61 g, 31%) as powders.
MS: 334 [M+H]⁺, APCI (MeOH)
(3) Compound D (2.61 g, 7.83 mmol) and 10% palladium carbon (0.26 g) were suspended in methanol (40 ml), and the mixture was stirred under hydrogen atmosphere at room temperature overnight. Insolubles were filtered off, and the filtrate was concentrated under reduced pressure. The residue was purified by NH silica gel column chromatography (hexane:ethyl acetate=95:5→75:25) to give Compound E (1.87 g, 91%) as powders.
MS: 244 [M+H]⁺, APCI (MeOH)

(4) Compound E was subjected to trifluoromethanesulfonylation according to Example 83(2), then, phenyl group was introduced according to Method 1, and the ester group was hydrolyzed according to the conventional manner, and subsequently, according to the manner of Method 7(B), Compound F was obtained.

MS: 275 [M+H]⁺, APCI (MeOH)

### Examples 85 to 126

The following compounds were obtained according to the methods and Examples described in the present specification, and the methods disclosed in the conventionally known literatures.

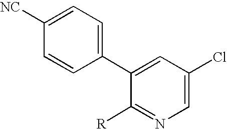

| Example | R | MS |
|---|---|---|
| 85 | 4-methylphenyl | 305/307 [M + H]⁺, APCI |
| 86 | 3-pyridyl | 292/294 [M + H]⁺, APCI |
| 87 | 4-fluorophenyl | 309/311 [M + H]⁺, APCI |
| 88 | 4-methoxyphenyl | 321/323 [M + H]⁺, APCI |
| 89 | 3-methylphenyl | 305/307 [M + H]⁺, APCI |
| 90 | 2-methylphenyl | 305/307 [M + H]⁺, APCI |
| 91 | 4-N,N-dimethylaminophenyl | 334/336 [M + H]⁺, APCI |
| 92 | 1-cyclohexenyl | 295/297 [M + H]⁺, APCI |
| 93 | 4-trifluoromethylphenyl | 359/361 [M + H]⁺, APCI |
| 94 | 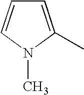 | 294/296 [M + H]⁺, APCI |
| 95 | 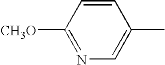 | 322/324 [M + H]⁺, APCI |
| 96 | 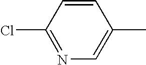 | 326/328 [M + H]⁺, APCI |
| 97 | 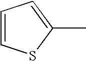 | 297/299 [M + H]⁺, APCI |
| 98 | 2-methoxyphenyl | 321/323 [M + H]⁺, APCI |
| 99 | 3-quinolyl | 342/344 [M + H]⁺, APCI |

### Example 100

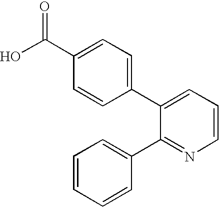 MS; 274 [M−H]⁻, ESI

### Example 101

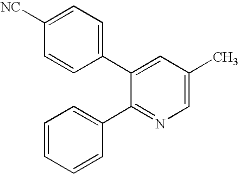

MS: 271 [M+H]⁺, APCI

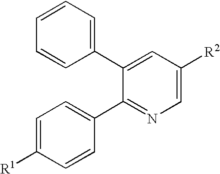

| Example | R¹ | R² | MS |
|---|---|---|---|
| 102 | NC— | Cl | 291/293 [M + H]⁺, APCI |
| 103 | HOOC— | Cl | 308/310 [M − H]⁻, ESI |
| 104 | NC— | H | 257 [M + H]⁺, APCI |
| 105 | HOOC— | H | 274 [M − H]⁻, ESI |

### Example 106

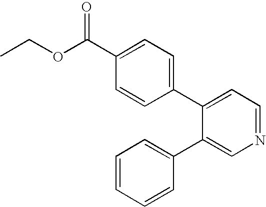

MS: 304 [M+H]⁺, APCI

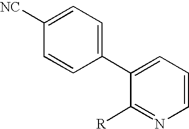

| Example | R | MS |
|---|---|---|
| 107 | 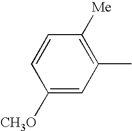 | 301 [M + H]⁺, APCI |
| 108 | 1-cyclohexenyl | 261 [M + H]⁺, APCI |
| 109 | 4-N,N-dimethylaminophenyl | 300 [M + H]⁺, APCI |
| 110 | 2-methoxyphenyl | 287 [M + H]⁺, APCI |
| 111 | 4-trifluoromethylphenyl | 325 [M + H]⁺, APCI |
| 112 | 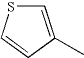 | 263 [M + H]⁺, APCI |
| 113 | 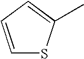 | 263 [M + H]⁺, APCI |
| 114 | 2-methylphenyl | 271 [M + H]⁺, APCI |
| 115 | 3-methylphenyl | 271 [M + H]⁺, APCI |
| 116 | 4-methoxyphenyl | 287 [M + H]⁺, APCI |
| 117 | 4-methylphenyl | 271 [M + H]⁺, APCI |
| 118 | 4-fluorophenyl | 275 [M + H]⁺, APCI |
| 119 | 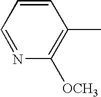 | 288 [M + H]⁺, APCI |

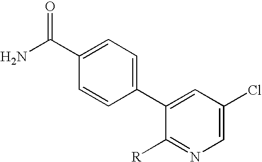

| Example | R | MS |
|---|---|---|
| 120 | 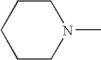 | 298/300 [M + H]⁺, APCI |
| 121 | (CH₃)₂CHNH— | 272/274 [M + H]⁺, APCI |
| 122 | 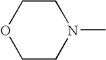 | 300/302 [M + H]⁺, APCI |

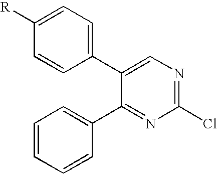

| Example | R | MS |
|---|---|---|
| 123 | HOOC— | 309/311 [M − H]⁻, ESI |
| 124 | H₂NOC— | 310/312 [M + H]⁺, APCI |

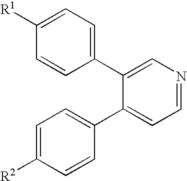

| Example | R¹ | R² | MS |
|---|---|---|---|
| 125 | H— | HOOC— | 274 [M − H]⁻, ESI |
| 126 | HOOC— | H— | 274 [M − H]⁻, ESI |

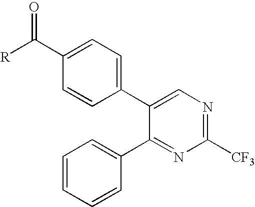

| Example | R | MS |
|---|---|---|
| 127 | HOCH₂CH₂NH— | 388 [M + H]⁺, APCI |
| 128 | (R)-CH₃CH(OH)CH₂NH— | 402 [M + H]⁺, APCI |
| 129 | (HOCH₂)₂CHNH— | 418 [M + H]⁺, APCI |
| 130 | (S)-HOCH₂CH(OH)CH₂NH— | 418 [M + H]⁺, APCI |
| 131 | (HOCH₂CH₂)₂NH— | 432 [M + H]⁺, APCI |
| 132 | H₂NCOCH₂NH— | 401 [M + H]⁺, APCI |

### Example 133

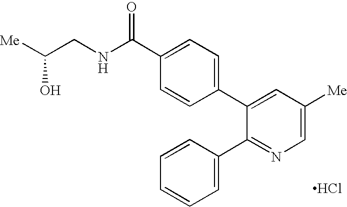 MS; 347 [M+H]⁺, APCI

### Example 134

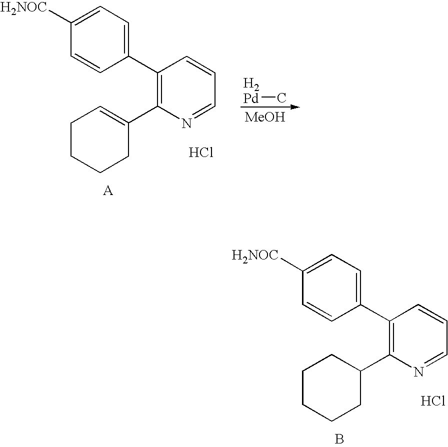

A solution of Compound A (32 mg, 0.102 mmol) and 10% Pd—C (6 mg) in methanol (2 ml) was stirred at room temperature under hydrogen atmosphere for 1 day. Then, the catalyst was removed by filtration and the filtrate was concentrated under reduced pressure. The residue was purified with preparative TLC (NH—SiO₂, chloroform:methanol=20:1) and treated with HCl to give Compound B (22 mg, 68%) as powders.

MS: 281 [M+H]⁺, APCI

### Example 135

The compound described in Example 22 can be obtained with good yield by the method as mentioned below.

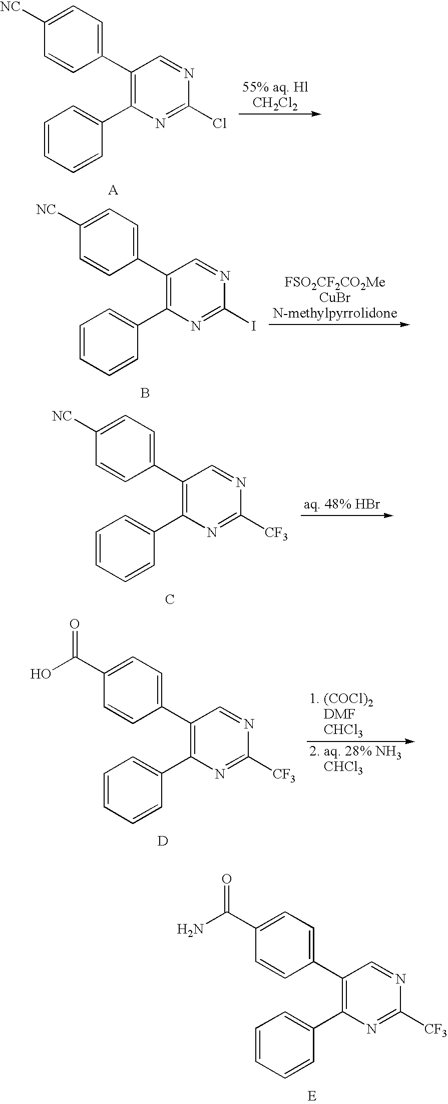

(1) To a solution of Compound A (1.50 g, 5.14 mmol) dissolved in CH₂Cl₂ (20 ml) was added dropwise an aqueous 55% hydriodic acid solution (15 ml, 107 mmol) at 0° C. under argon atmosphere over a period of 15 minutes. After completion of addition, the mixture was stirred at the same temperature for 30 hours. A saturated aqueous sodium bicarbonate solution and an aqueous 10% sodium sulfite solution were added to the mixture, successively. The mixture was extracted with chloroform. The organic layer was dried over anhydrous sodium sulfate, and concentrated under reduced pressure. The residue was purified by silica gel column chromatography (hexane:ethyl acetate=90:10→80:20) to give a mixture of Compounds B and A (1.16 g, mol ratio 87:13) as powders.

MS: 384 [M+H]⁺, APCI (MeOH)

(2) The obtained mixture of Compounds B and A (1.16 g, mol ratio 87:13), methyl fluorosulfonyl(difluoro)acetate (775 μl, 6.09 mmol), and copper(I) bromide (87 mg, 0.61 mmol) were stirred in NMP (30 ml) at 120° C. under argon atmosphere for 3 hours. After cooling the reaction mixture, ethyl acetate was added thereto and the organic layer was washed with water and brine, dried over anhydrous sodium sulfate, and concentrated under reduced pressure. The residue was purified by silica gel column chromatography (hexane:ethyl acetate=90:10) to give Compound C (0.744 g, 44% for 2 steps) as powders.

MS: 326 [M+H]⁺, APCI (MeOH)

(3) Compound C (0.449 g, 1.38 mmol) was added to an aqueous 48% hydrobromic acid solution (15 ml), and the suspension was refluxed under argon atmosphere for 8 hours. After cooling the reaction mixture, diethylether was added thereto and the organic layer was extracted with 2M aqueous sodium hydroxide solution. The aqueous layer, after 36% hydrochloric acid was added thereto until it was adjusted to pH 1, was extracted with a mixed solution of methanol-chloroform (9:1). The organic layer was dried over anhydrous magnesium sulfate, and concentrated under reduced pressure to afford Compound D (0.478 g, 100%) as a solid. MS: 343 [M−H]⁻, ESI (MeOH)

(4) To a suspension of Compound D (80 mg, 0.232 mmol) in chloroform (5.0 ml) were added oxalyl chloride (61 ul, 0.699 mmol) and N,N-dimethylformamide (1 drop), and the mixture was stirred at room temperature for 30 minutes. The mixture was concentrated under reduced pressure to give acid chloride. To an aqueous 28% ammonia (3.0 ml, 49 mmol) was added the suspension of obtained acid chloride in chloroform (3.0 ml) at 0° C. over a period of 3 minutes. After completion of addition, the mixture was stirred at the same temperature for 30 minutes. Water was added thereto and the mixture was extracted with a mixed solution of methanol-chloroform(9:1). The organic layer was dried over anhydrous sodium sulfate, and concentrated under reduced pressure. The residue was purified by silica gel column chromatography (chloroform:methanol=100:0→90:10) to give Compound E (45 mg, 57%) as powders.

MS: 344 [M+H]⁺, APCI (MeOH)

### Reference Examples 1 to 4

The following compounds were obtained as synthetic intermediates according to the methods and Examples described in the present specification, and the methods disclosed in the conventionally known literatures.

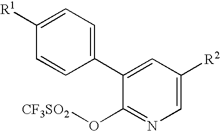

| Reference | | | |
|---|---|---|---|
| example | R¹ | R² | MS |
| 1 | NC— | H— | 346 [M + NH₄]⁺, APCI |
| 2 | NC— | Cl— | — |
| 3 | H— | Cl— | — |
| 4 | H— | H— | 304 [M + H]⁺, APCI |

### Experimental Example 1

[Relaxation Effect on Potassium-Induced Contraction of Isolated Rabbit Urinary Bladder]

Urinary bladder was isolated from Male NZW rabbits (body weight: 2.0-3.5 kg) and immersed in ice-cold Krebs-bicarbonate solution (in mM: 118 NaCl, 4.7 KCl, 2.55 CaCl₂, 1.18 MgSO₄, 1.18 KH₂PO₄, 24.88 NaHCO₃ and 11.1 glucose). The urinary bladder was cut into longitudinal strips (5 mm length, 3-4 mm width) after mucosal layer was removed.

Preparations were mounted in organ baths containing 10 ml of Krebs solution maintained at 37° C. and gassed with 95% O₂/5% CO₂. Accordingly, preparations were stretched with an initial tension of 2.0±1.0 g, and changes in isometric tension were measured by force-displacement transducer. The preparations were pre-contracted by changing organ-bath solution into high-K⁺ (30 mM) Krebs solution (in mM: 118 NaCl, 4.7 KCl, 2.55 CaCl₂, 1.18 MgSO₄, 1.18 KH₂PO₄, 24.88 NaHCO₃ and 11.1 glucose).

After stable tension was obtained, compounds were added into organ baths cumulatively (10⁻⁸ M-10⁻⁴ M). The effects of compounds were expressed as a percentage of the maximum relaxation produced by 10⁻⁴ M papaverine as 100%. 50% relaxation concentration (IC₅₀) was calculated and IC₅₀ value range (μM) of compounds of the present invention was shown in the following Table 1 with a rank of A, B or C.

These ranges are as mentioned below.

3 μM≧C>1 μM≧B>0.5 μM≧A

TABLE 1

| Test compound | IC₅₀ value |
|---|---|
| Example 1 | B |
| Example 8 | C |
| Example 13 | C |
| Example 16 | B |
| Example 43 | B |

### Experimental Example 2

[Inhibitory Effect on the Rhythmic Bladder Contractions Induced by Substance P in Anesthetized Rats]

For the experiments, Sprague-Dawley female rats (9 to 12 weeks old) weighing between 200 to 300 g were used. After urethane anesthetization (subcutaneously administered with a dose of 1.2 g/kg), cannulae were placed in both right and left femoral veins. One intravenous catheter was used for administration of compounds, and the other was for the substance P (0.33 μg/kg/min) infusion. We also cannulated into ureter to pass urine. Polyethylene catheters were inserted into carotid artery for continuous monitoring of arterial blood pressure and heart rate. For continuous infusion, transurethral bladder catheter was inserted into the bladder through the urethra and tied in place by a ligature around the urethral orifice. One end of the catheter was attached to a pressure transducer in order to measure intravesical pressure. The other end of the catheter was used for infusion of saline into the bladder. After stabilization of blood pressure and heart rate and after the bladder was emptied, cystometry was performed by filling the bladder slowly with about 0.6 ml of saline. After about 10 minutes, intravenous infusion of substance P (0.33 μg/kg/min) was started for stabilization of the micturition reflex. Compounds were administered after stable rhythmic bladder contraction was obtained over 15 minutes. All compounds were dissolved or suspended in saline containing 0.5% Tween 80 for intravenous administration (0.1 ml/kg). The rhythmic contraction frequency and the intravesical pressure were observed for 35 minutes after administration of the test compound.

As a result, compounds of the present invention decreased the frequency of bladder rhythmic contraction without changing the amplitude of contraction. Also, we determined a time (minute) during which the frequency of the rhythmic contraction had been completely inhibited by administering 0.25 mg/kg of compound. A 100% inhibition time (minute) of the selected compounds of the present invention is shown in the following Table 2.

TABLE 2

| Test compound | Time (min) |
|---|---|
| Example 1 | 20.5 |
| Example 8 | 18 |
| Example 13 | 26.8 |
| Example 43 | >35 |

Also, pre-administration of iberiotoxin a selective large conductance calcium-activated K channel blocker (0.15 mg/kg, intravenous administration) reduced inhibitory effect of the compound of the present invention on the rhythmic bladder contraction. Thus, it is suggested that the tricyclic compounds of the present invention have a detrusor relaxing activity through the large conductance calcium-activated K channel, and were effective for prophylaxis and treatment of diseases such as pollakiuria, urinary incontinence and the like through the large conductance calcium-activated K channel opening activity.

### INDUSTRIAL APPLICABILITY

The bicyclic compound of the present invention has an excellent large conductance calcium-activated K channel opening activity, so that it is useful for a prophylactic, relief and/or treatment agent of, for example, pollakiuria, urinary incontinence, asthma, COPD, and the like.

1. A bicyclic compound of formula (1): 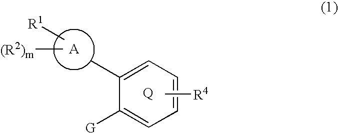
wherein Ring Q is pyridine or pyrimidine;
Ring A is benzene or a heteroaromatic ring;
G is 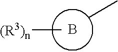
or an amino optionally substituted by one or two selected from the group consisting of alkyl(s), aralkyl(s) and cycloalkyl(s);
Ring B is benzene, a heterocyclic ring, a cycloalkane or a cycloalkene;
R¹ is a group selected from the following formulae: 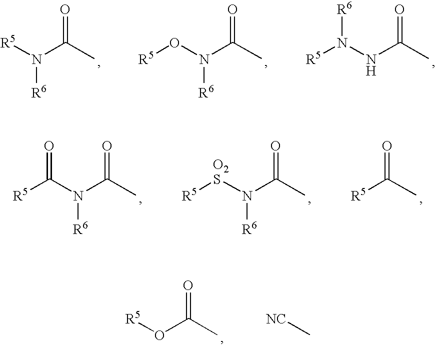
R² and R³ may be the same or different from each other, and each is cyano, nitro, hydroxyl, an alkoxy, a halogen, carboxyl, an alkoxycarbonyl, an optionally substituted carbamoyl, an optionally substituted amino or an optionally substituted alkyl; provided that when m is 2, two R²s may be the same or different from each other, and when n is 2, two R³s may be the same or different from each other;
m and n may be the same or different from each other, and each is 0, 1 or 2;
R⁴ is hydrogen, a halogen, cyano, an alkoxy, hydroxyl, carbamoyl, an optionally substituted amino, an optionally substituted alkyl, an optionally substituted aryloxy, a cycloalkyloxy or an optionally substituted heterocyclic group; and
R⁵ and R⁶ may be the same or different from each other, and each is hydrogen, an optionally substituted alkyl, an optionally substituted cycloalkyl where the cycloalkyl may be fused with an aryl, an optionally substituted aryl, an optionally substituted heterocyclic group, or an alkoxycarbonyl, or R⁵ and R⁶ may form an optionally substituted heterocyclic ring in combination with atoms to which they are bonded, excluding 4-amino-5-(4-cyanophenyl)pyrimidine,
or a pharmaceutically acceptable salt thereof.

2. The bicyclic compound or a pharmaceutically acceptable salt thereof according to claim 1, which compound is a compound of (1a): 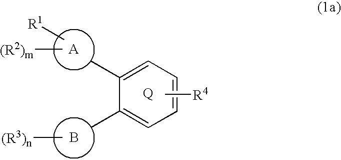
wherein Ring Q, Ring A, Ring B, R¹, R², R³, R⁴, m and n have the same meanings as defined in claim 1.

3. The bicyclic compound or a pharmaceutically acceptable salt thereof according to claim 1 or 2, wherein the Ring Q is pyridine.

4. The bicyclic compound or a pharmaceutically acceptable salt thereof according to claim 1, which compound is a compound of (1b): 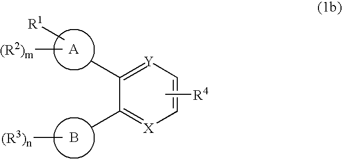
wherein one of X and Y is nitrogen, and the other is methine; Ring A, Ring B, R¹, R², R³, R⁴, m and n have the same meanings as defined in claim 1.

5. The bicyclic compound or a pharmaceutically acceptable salt thereof according to claim 1, wherein Ring A is a 5- or 6-membered ring.

6. The bicyclic compound or a pharmaceutically acceptable salt thereof according to claim 1, wherein the Ring A is benzene, pyridine, pyrimidine or thiophene.

7. The bicyclic compound or a pharmaceutically acceptable salt thereof according to claim 1, wherein the Ring A is benzene.

8. The bicyclic compound or a pharmaceutically acceptable salt thereof according to claim 1, wherein Ring A is a 6-membered ring and R¹ is bonded to Ring A at the para-position to Ring Q.

9. The bicyclic compound or a pharmaceutically acceptable salt thereof according to claim 1, wherein Ring B is benzene, pyridine, pyrimidine, thiophene, piperidine, morpholine, cyclohexane, cyclohexene, pyrrolidine or pyrrole.

10. The bicyclic compound or a pharmaceutically acceptable salt thereof according to claim 1, wherein R¹ is a group selected from the following formulae:

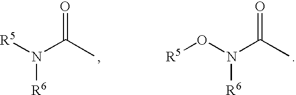

11. The bicyclic compound or a pharmaceutically acceptable salt thereof according to claim 10, wherein R⁵ is hydrogen, an optionally substituted alkyl wherein substituent(s) for the substituted alkyl are 1 to 3 groups selected from the following formulae, an optionally substituted cycloalkyl where said cycloalkyl may be fused with an aryl, an optionally substituted aryl or an optionally substituted heterocyclic group, and R⁶ is hydrogen, an alkoxycarbonyl, or an alkyl optionally substituted by hydroxyl(s) or alkoxy(s), or R⁵ and R⁶ may form an optionally substituted heterocyclic ring in combination with atom(s) to which they are bonded, 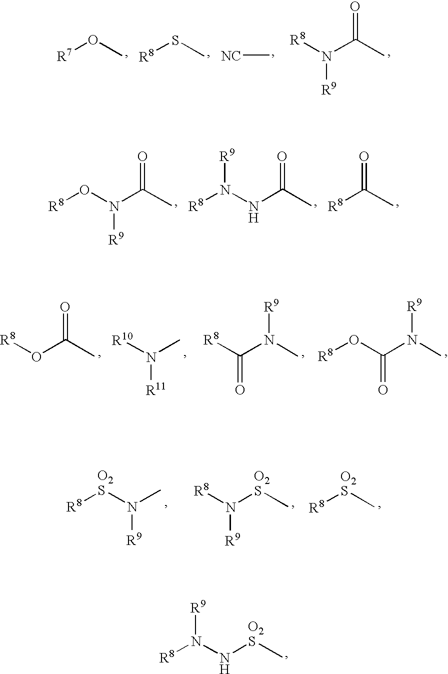
optionally substituted heterocyclic group, optionally substituted aryl,
wherein R⁷ is (1) hydrogen, (2) an alkyl which may be optionally substituted by an optionally substituted aryl or an optionally substituted heterocyclic group, (3) a hydroxyalkyl, (4) an alkoxyalkyl or (5) an optionally substituted heterocyclic group;
R⁸ and R⁹ may be the same or different from each other, and each is (1) hydrogen, (2) an alkyl optionally substituted by an optionally substituted aryl or an optionally substituted heterocyclic group, (3) a hydroxyalkyl, (4) an alkoxyalkyl, (5) an alkoxycarbonyl, (6) an optionally substituted heterocyclic group or (7) an optionally substituted aryl or (8) R⁸ and R⁹ may form an optionally substituted heterocyclic ring in combination with atoms to which they are bonded; and
R¹⁰ and R¹¹ may be the same or different from each other, and each is (1) hydrogen, (2) an alkyl optionally substituted by an optionally substituted aryl or an optionally substituted heterocyclic group, (3) a hydroxyalkyl, (4) an alkoxyalkyl, (5) an alkanoyl, (6) an alkylsulfonyl, (7) an alkoxycarbonyl or (8) an optionally substituted heterocyclic group.

12. The bicyclic compound or a pharmaceutically acceptable salt thereof according to claim 11, wherein the substituent(s) for the substituted alkyl of R⁵ are 1 to 3 groups selected from the following formulae: 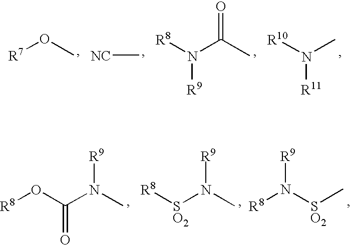
optionally substituted heterocyclic group, optionally substituted aryl,
wherein R⁷, R⁸, R⁹, R¹⁰ and R¹¹ have the same meanings as defined in claim 11.

13. The bicyclic compound or a pharmaceutically acceptable salt thereof according to claim 11, wherein the substituent(s) for the substituted alkyl of R⁵ are 1 to 3 groups selected from the following formulae: 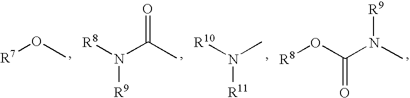
optionally substituted heterocyclic group, optionally substituted aryl,
wherein R⁷, R⁸, R⁹, R¹⁰ and R¹¹ have the same meanings as defined in claim 11.

14. The bicyclic compound or a pharmaceutically acceptable salt thereof according to claim 1, wherein Ring Q is pyrimidine and R¹ is a group selected from the following formulae: 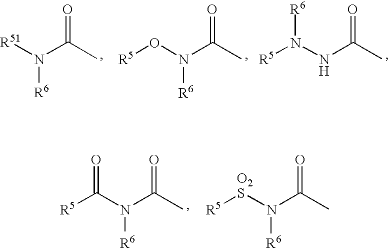
wherein R⁵ and R⁶ have the same meanings as defined in claim 1, R⁵¹ is an alkyl substituted by 1 to 3 groups selected from the following formulae: 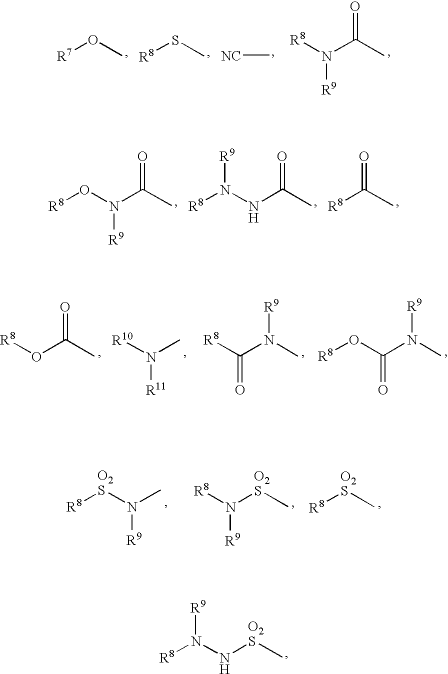
wherein R⁷, R⁸, R⁹, R¹⁰ and R¹¹ have the same meanings.

15. The bicyclic compound or a pharmaceutically acceptable salt thereof according to claim 1, wherein m and n may be the same or different from each other, and each is 0 or 1.

16. The bicyclic compound or a pharmaceutically acceptable salt thereof according to claim 1, wherein R² and R³ may be the same or different from each other, and each is cyano, hydroxyl, alkoxy, a halogen or optionally substituted alkyl.

17. The bicyclic compound or a pharmaceutically acceptable salt thereof according to claim 1, wherein R⁴ is hydrogen, a halogen, or optionally substituted alkyl.

18. A medicine comprising the bicyclic compound or a pharmaceutically acceptable salt thereof according to claim 1.

19. The medicine according to claim 18, which is a large conductance calcium-activated K channel opener.

20. The medicine according to claim 18, which is for the prophylaxis and/or treatment of pollakiuria, urinary incontinence, asthma or chronic obstructive pulmonary diseases.

21. The medicine according to claim 20, which is for the prophylaxis and/or treatment of pollakiuria, urinary incontinence or chronic obstructive pulmonary diseases.
